(19) United States
(12) Patent Application Publication
(10) Pub. No.: US 2016/0232293 A1
(43) Pub. Date: Aug. 11, 2016

(54) DRUG SENSITIVITY BIOMARKERS AND METHODS OF IDENTIFYING AND USING DRUG SENSITIVITY BIOMARKERS

(71) Applicant: SANFORD-BURNHAM MEDICAL RESEARCH INSTITUTE, LaJolla, CA (US)

(72) Inventors: Adam Godzik, LaJolla, CA (US); Eduardo Porta-Pardo, LaJolla, CA (US)

(21) Appl. No.: 15/029,676

(22) PCT Filed: Oct. 17, 2014
(86) PCT No.: PCT/US2014/061182

§ 371 Date: Apr. 15, 2016

Related U.S. Application Data
(60) Provisional application No. 61/892,293, filed on Oct. 17, 2013.

(51) Int. Cl. G06F 19/24; G01N 33/68

(52) U.S. Cl. CPC G06F 19/24; G01N 33/68; G01N 2570/00

(57) ABSTRACT

Disclosed are methods based on correlation of drug effects with genetic alterations in specific sub-regions of proteins. The presence of such genetic alterations in subjects with a relevant disease allows more directed treatment of the disease, ideally limited to subjects having a genetic alteration in the drug effect-correlated sub-region of a protein. Disclosed are methods of identifying subjects, treating subjects, identifying specific drug effect-correlated protein sub-regions, and identifying drugs correlated with specific protein sub-regions, all based on the discovered correlation of drug effects with genetic alterations in specific sub-regions of proteins.

### CROSS-REFERENCE TO RELATED APPLICATIONS

This application claims benefit of U.S. Provisional Application No. 61/892,293, filed Oct. 17, 2014.

### FIELD OF THE INVENTION

The disclosed invention is generally in the field of analysis of protein mutants and variants and specifically in the area of analysis of correlation of protein variants with phenotypes, such as dug effects.

### BACKGROUND OF THE INVENTION

With the body of genomic and pharmacologic data on cancer growing exponentially, the main bottleneck to translate such information into meaningful and clinically relevant hypothesis is data analysis (Barretina et al., Nature 483:603-607 (2012); Yang et al., Nucleic Acids Res 41:D955-961 (2013); Good et al., Genome Biology 15:438 (2014)). While numerous methods have been recently applied to the analysis of such datasets (Jerby-Arnon et al., Cell 158:1199-1209 (2014)) most of them, particularly those dealing with mutation data (Costello et al., Nat Biotechnol doi:10.1038/nbt.2877 (2014)), use a protein-centric perspective, as they do not take into account the specific position of the different mutations within a protein (Basu et al., Cell 154:1151-1161 (2013); Mo et al., Proc Natl Acad Sci USA 110:6 (2013)). Such approaches have been proven useful in many applications; however, they cannot fully deal with situations in which different mutations in the same protein have different effects depending on which region of the protein is being altered (Kobayashi et al., New England Journal of Medicine 325:7 (2005)).

It has been discovered that such protein-centric analyses of genetic alterations miss subtler, yet still relevant, effects mediated by mutations in specific protein regions. The solution to the problems in protein-centric analysis was discovered to be in the analysis of perturbations in specific protein regions and correlating such region-level perturbations with drug effects. This provides richer and more effective information on drugs and their effects on cancer.

Accordingly, it is an object of the present invention to provide methods of identifying subjects having specific drug effect-correlated protein sub-regions.

It is a further object of the present invention to provide methods of treating subjects having specific drug effect-correlated protein sub-regions.

It is a further object of the present invention to provide methods of identifying specific drug effect-correlated protein sub-regions.

It is a further object of the present invention to provide methods of identifying drugs correlated with specific protein sub-regions.

### SUMMARY OF THE INVENTION

It has been discovered that genetic alterations in specific subsections or regions of proteins can be correlated with drug effects and the associated diseases when genetic alterations averaged over the protein as a whole do not show such a correlation. This discovery permits an expansion in genetic features that have relevance to and uses in treating disease. The genetic features can have a positive effect (e.g., where a mutation makes a cell susceptible to a drug) or a negative effect (e.g., where a mutation makes a cell resistant to a drug). The presence or absence of a genetic alteration can thus have either a positive or negative effect. One type of protein subsection that has relevance to the present discovery is protein functional region (PFR or plural, PFRs). PFRs include functional domains of a protein and intrinsically disordered regions (IDRs) of the protein. Genetic features grouped by PFR, PFR group (i.e., two or more, but fewer than all, of the PFRs in a protein), whole protein, and sets of any combination of these “protein units” can be used as potential correlates to drug effects and diseases.

Disclosed are methods based on correlation of drug effects with genetic alterations in specific sub-regions of proteins. The presence of such genetic alterations in subjects with a relevant disease allows more directed treatment of the disease, ideally limited to subjects having a genetic alteration in the drug effect-correlated sub-region of a protein. Disclosed are methods of identifying subjects, treating subjects, identifying specific drug effect-correlated protein sub-regions, and identifying drugs correlated with specific protein sub-regions, all based on the discovered correlation of drug effects with genetic alterations in specific sub-regions of proteins.

Disclosed are methods of treating a disease by treating a subject having the disease and identified as having genetic features in a drug-specific set of protein units with a compound identified as a protein unit-specific compound for the drug-specific set of protein units. Protein units include PFRs, PFR groups, and whole proteins. A drug-specific set of protein units is a set of protein units where genetic features in the set of protein units are correlated with an effect of the compound. A protein unit-specific compound is a compound an effect of which is correlated the presence of a genetic feature in a protein unit or genetic features in a set of protein units.

The disease can be a protein unit-associated disease for the drug-specific set of protein units. A protein unit-associated disease is a disease for which a drug-specific protein unit or drug-specific set of protein units is correlated with is correlated with an effect of a compound on the disease. Such an effect (i.e., an effect involved in such a correlation) is a disease-associated effect for the disease. Similarly, the compound involved in such a correlation is a disease-associated compound for the disease.

In some forms of the methods, at least one of the protein units in the drug-specific set of protein units is a PFR or a PFR group of a protein, where genetic features in the PFR or PFR group of the protein are correlated with an effect of the compound but where genetic features in the protein as a whole are not correlated with the effect of the compound.

In some forms of the methods, the set of protein units can consist of a single PFR for a protein. In some forms of the methods, the disease is cancer, the disease-associated effect is an anticancer effect, and the genetic features in the drug-specific set of protein units are present in one or more cancer cells of the subject. In some forms of the methods, the subject is identified as having one or more cells having the genetic features in the drug-specific set of protein units prior to treatment. In some forms of the methods, the genetic features are detected in the drug-specific set of protein units in one or more cells of the subject prior to treatment. In some forms of the methods, the cells are disease-related cells for the disease. A disease-related cell for a disease is a type of cell of which some genetic alterations are correlated with a disease. For example, cancer cells are disease-related cells for cancer. Generally, disease-related cells are cells involved in the disease. But genetic features can be present in non-involved cells (such as when a subject's cells contain a disease-predisposing genetic alteration).

Also disclosed are methods of identifying a drug-specific set of protein units for a compound and a disease by assessing correlation between genetic features in a test set of protein units and the effect of a compound on a disease, where identification of a correlation between genetic features in the test set of protein units and the effect of the compound on a disease identify the test set of protein units as a drug-specific set of protein units for the compound and for the disease and identify the compound as a protein unit/disease-associated compound for the disease and for the test set of protein units. A protein unit/disease-associated compound is a compound an effect of which on the disease is correlated with the presence of a genetic feature in a protein unit or genetic features in a set of protein units. In some forms of the method, at least one of the protein units in the test set of protein units is a PFR or a PFR group of a protein

Also disclosed are methods of identifying protein unit-specific compounds for a set of protein units and a disease by assessing correlation between genetic features in a set of protein units and the effect of a test compound on a disease, where identification of a correlation between genetic features in the set of protein units and the effect of the test compound on a disease identify the test compound as a protein unit-specific compound for the set of protein units and for the disease and identify the set of protein units as a drug-specific set of protein units for the disease and for the test compound.

In some forms of the methods, the test set of protein units can include at least one PFR and at least one whole protein. In some forms of the methods, the test set of protein units can include at least two PFRs. In some forms of the methods, the test set of protein units can include at least one PFR group.

In some forms of the methods, the test set of protein units can consist of a single PFR for a protein and the method further comprises assessing correlation between genetic features of the protein as a whole and the effect of the compound on the disease, where identification of a correlation between genetic features in the PFR for the protein and the effect of the compound on a disease and a lack of correlation between genetic features of the protein as a whole and the effect of the compound on the disease identify the PFR of the protein as a drug-specific PFR for the compound and for the disease and identify the compound as a PFR/disease-associated compound for the disease and for the PFR of the protein.

In some forms of the methods, the set of protein units can consist of a single PFR for a protein and the method further comprises assessing correlation between genetic features of the protein as a whole and the effect of the test compound on the disease, where identification of a correlation between genetic features in the PFR of the protein and the effect of the test compound on a disease and a lack of correlation between genetic features of the protein as a whole and the effect of the test compound on the disease identify the test compound as a PFR-specific compound for the PFR of the protein and for the disease and identify the PFR of the protein as a drug-specific PFR for the disease and for the test compound.

In some forms of the methods, identification of the correlations can be accomplished by identifying protein units in proteins, categorizing genetic features by protein unit, where the genetic features are present or not present in disease-related cells, categorizing the genetic features by whether the compound has the effect on the disease in subjects having the disease and having the genetic features or by whether the compound has the effect on the disease-related cells affected by the disease and having the genetic features, and calculating the level of correlation between genetic features in the protein units and the effect of the compound.

In some forms of the methods, the method can further comprise calculating the level of correlation between genetic features in proteins as a whole and the effect of the compound. In some forms of the methods, the disease-related cells are cancer cell lines and the genetic features are categorized by whether the compound has the effect on the cancer cell lines having the genetic features.

Also disclosed are methods of contributing to improving the effectiveness of a treatment of a disease in a population of subjects that have the disease by treating a subject having genetic features in a drug-specific set of protein units in one or more disease-related cells with a protein unit-specific compound for the set of protein units and for the disease and refraining from treating a subject that does not have genetic features in one or more members of the drug-specific set of protein units of one or more disease-related cells with the protein unit-specific compound. The drug-specific set of protein units is a set of protein units where genetic features in the set of protein units are correlated with an effect of the compound, the effect is a disease-associated effect for the disease, the compound is a disease-associated compound for the disease, and the disease is a protein unit-associated disease for the drug-specific set of protein units.

In some forms of the methods, at least one of the protein units in the drug-specific set of protein units is a PFR or a PFR group of a protein, where genetic features in the PFR or PFR group of the protein are correlated with an effect of the compound but where genetic features in the protein as a whole are not correlated with the effect of the compound.

In some forms of the methods, the set of protein units can consist of a single PFR for a protein. In some forms of the methods, the disease is cancer, the disease-associated effect is an anticancer effect, and the genetic features in the drug-specific set of protein units is present in one or more cancer cells of the subject. In some forms of the methods, the subject is identified as having one or more cells having the genetic features in the drug-specific set of protein units prior to treatment. In some forms of the methods, the genetic features are detected in the drug-specific set of protein units in one or more cells of the subject prior to treatment. In some forms of the methods, the cells are disease-related cells for the disease.

Also disclosed are methods of treating cancer by treating a subject having cancer and identified as having a genetic feature in a drug-specific PFR with a PFR-specific compound for the drug-specific PFR, wherein the drug-specific PFR and PFR-specific compound for the drug-specific PFR are selected from one of the pairs in Table 1.

TABLE 1

| Drug-Specific PFR | Compound |
|---|---|
| Amino acids 1245 to 1508 of MAP3K1 | Lapatinib |
| Amino acids 1246 to 1503 of MAP3K1 | Lapatinib |
| Amino acids 123 to 407 of MSH6 | AEW541 |
| Amino acids 280 to 460 of CACNB2 | L-685458 |
| Amino acids 148 to 248 of ADAM22 | TKI258 |
| Amino acids 1818 to 2102 of TPR | ZD-6474 |
| Amino acids 334 to 699 of AFF4 | PD-0325901 |
| Amino acids 76 to 288 of HDAC4 | Sorafenib |
| Amino acids 137 to 218 of PRKG1 | Sorafenib |
| Amino acids 38 to 151 of DAPK1 | PHA-665752 |
| Amino acids 1221 to 1309 of ITGB4 | TAE684 |
| Amino acids 2514 to 2657 of LAMA1 | AEW541 |
| Amino acids 2514 to 2653 of LAMA1 | AEW541 |
| Amino acids 28254 to 28339 of TTN | Topotecan |
| Amino acids 1442 to 1492 of MTOR | Topotecan |
| Amino acids 520 to 703 of PIK3CA | AEW541 |
| Amino acids 252 to 322 of DAPK1 | PLX4720 |
| Amino acids 814 to 1266 of SETDB1 | PF2341066 |
| Amino acids 814 to 1266 of SETDB1 | TAE684 |
| Amino acids 2514 to 2657 of LAMA1 | PF2341066 |
| Amino acids 2514 to 2653 of LAMA1 | PF2341066 |
| Amino acids 644 to 733 of DPYD | TKI258 |
| Amino acids 172 to 406 of MAP3K13 | RAF265 |
| Amino acids 171 to 406 of MAP3K13 | RAF265 |
| Amino acids 190 to 442 of TNK2 | TKI258 |
| Amino acids 4468 to 4599 of LRP1B | Sorafenib |
| Amino acids 748 to 903 of CDH2 | 17-AAG |
| Amino acids 1846 to 2050 of PI4KA | PD-0325901 |
| Amino acids 1818 to 2102 of TPR | TKI258 |
| Amino acids 980 to 1244 of INSRR | PD-0332991 |
| Amino acids 980 to 1244 of INSRR | PD-0332991 |
| Amino acids 28254 to 28339 of TTN | Lapatinib |
| Amino acids 60 to 233 of EPHA5 | Nutlin-3 |
| Amino acids 334 to 699 of AFF4 | AZD6244 |
| Amino acids 1 to 68 of MYC | AZD0530 |
| Amino acids 1345 to 1639 of CREBBP | AZD6244 |
| Amino acids 667 to 923 of PAPPA | LBW242 |
| Amino acids 28254 to 28339 of TTN | Nilotinib |
| Amino acids 979 to 1119 of CLTCL1 | TAE684 |
| Amino acids 32 to 108 of PIK3CA | AEW541 |
| Amino acids 816 to 1002 of GUCY2C | PHA-665752 |
| Amino acids 76 to 288 of HDAC4 | TKI258 |
| Amino acids 897 to 1184 of MECOM | ZD-6474 |
| Amino acids 1068 to 1217 of BCR | TAE684 |
| Amino acids 1 to 172 of SMG1 | LBW242 |
| Amino acids 1044 to 1233 of TIAM1 | L-685458 |
| Amino acids 30721 to 30807 of TTN | RAF265 |
| Amino acids 4993 to 5069 of TTN | PF2341066 |
| Amino acids 4990 to 5059 of TTN | PF2341066 |
| Amino acids 1083 to 1222 of BIRC6 | Nutlin-3 |
| Amino acids 148 to 248 of ADAM22 | Nilotinib |
| Amino acids 279 to 373 of PPARGC1A | Panobinostat |
| Amino acids 1695 to 1822 of TG | Panobinostat |
| Amino acids 1 to 68 of MYC | TAE684 |
| Amino acids 2694 to 2748 of CSMD3 | PD-0325901 |
| Amino acids 32714 to 32792 of TTN | AZD0530 |
| Amino acids 1125 to 1280 of NCOA2 | Erlotinib |
| Amino acids 807 to 1069 of PTK7 | PD-0325901 |
| Amino acids 695 to 878 of ALS2 | Panobinostat |
| Amino acids 114 to 294 of CTTN | ZD-6474 |
| Amino acids 622 to 697 of TNN | AEW541 |
| Amino acids 586 to 808 of BAI3 | AZD0530 |
| Amino acids 134 to 413 of EXT2 | TAE684 |
| Amino acids 2971 to 3050 of TTN | Topotecan |
| Amino acids 26686 to 26766 of TTN | 17-AAG |
| Amino acids 60 to 162 of ADAM12 | Irinotecan |
| Amino acids 492 to 561 of CPNE5 | AZD0530 |
| Amino acids 274 to 367 of TSSK1B | TAE684 |
| Amino acids 561 to 794 of MSH5 | ZD-6474 |
| Amino acids 561 to 794 of MSH5-SAPCD1 | ZD-6474 |
| Amino acids 303 to 334 of TNNI3K | AEW541 |
| Amino acids 521 to 605 of PCDH15 | Irinotecan |
| Amino acids 2054 to 2236 of MLL3 | Lapatinib |
| Amino acids 3718 to 3754 of LRP2 | PLX4720 |
| Amino acids 737 to 1068 of UBE3B | Panobinostat |
| Amino acids 7795 to 7885 of TTN | Topotecan |
| Amino acids 280 to 460 of CACNB2 | AZD0530 |
| Amino acids 137 to 218 of PRKG1 | TAE684 |
| Amino acids 1916 to 2020 of NAV3 | 17-AAG |
| Amino acids 87 to 802 of MYH10 | TAE684 |
| Amino acids 220 to 389 of NLRP3 | PD-0332991 |
| Amino acids 1711 to 2049 of CNTRL | TAE684 |
| Amino acids 1409 to 1488 of TAF1L | Panobinostat |
| Amino acids 824 to 916 of PCDH15 | Nutlin-3 |
| Amino acids 817 to 925 of CUBN | Nilotinib |
| Amino acids 1224 to 1458 of PTPRT | Paclitaxel |
| Amino acids 1649 to 1795 of FANCM | Nutlin-3 |
| Amino acids 769 to 942 of RASA1 | PF2341066 |
| Amino acids 87 to 802 of MYH10 | AZD0530 |
| Amino acids 947 to 1234 of GRIN2A | AZD6244 |
| Amino acids 50 to 94 of PLCG1 | PHA-665752 |
| Amino acids 40 to 140 of PLCG1 | PHA-665752 |
| Amino acids 410 to 617 of ZNF608 | Lapatinib |
| Amino acids 807 to 1069 of PTK7 | AZD6244 |
| Amino acids 199 to 527 of HIPK2 | TKI258 |
| Amino acids 190 to 442 of TNK2 | Nutlin-3 |
| Amino acids 31 to 186 of ADAMTS20 | AZD0530 |
| Amino acids 914 to 1030 of AATK | Lapatinib |
| Amino acids 382 to 604 of PAXIP1 | RAF265 |
| Amino acids 538 to 699 of MSH6 | Lapatinib |
| Amino acids 555 to 638 of SMO | 17-AAG |
| Amino acids 75 to 408 of GUCY2F | LBW242 |
| Amino acids 249 to 426 of RASGRF2 | Paclitaxel |
| Amino acids 524 to 607 of ROBO2 | PHA-665752 |
| Amino acids 400 to 545 of ACOXL | AZD0530 |
| Amino acids 645 to 739 of GTSE1 | PF2341066 |
| Amino acids 1 to 68 of MYC | AZD6244 |
| Amino acids 190 to 442 of TNK2 | ZD-6474 |
| Amino acids 46 to 188 of ALK | Panobinostat |
| Amino acids 512 to 728 of GUCY1A2 | LBW242 |
| Amino acids 1256 to 1451 of NF1 | Panobinostat |
| Amino acids 1249 to 1465 of COL3A1 | PHA-665752 |
| Amino acids 1 to 87 of SRPK1 | Lapatinib |
| Amino acids 21 to 253 of URB2 | RAF265 |
| Amino acids 320 to 391 of PRKD3 | ZD-6474 |
| Amino acids 47 to 157 of INSRR | Lapatinib |
| Amino acids 712 to 924 of AFF4 | PD-0325901 |
| Amino acids 92 to 354 of ROCK2 | Nilotinib |
| Amino acids 573 to 1207 of MYO18B | Irinotecan |
| Amino acids 612 to 807 of RABEP1 | Nutlin-3 |
| Amino acids 118 to 147 of TEC | PF2341066 |
| Amino acids 2407 to 2475 of SPTAN1 | L-685458 |
| Amino acids 2743 to 2868 of LAMA1 | PD-0332991 |
| Amino acids 2743 to 2872 of LAMA1 | PD-0332991 |
| Amino acids 825 to 1090 of TEK | AZD0530 |
| Amino acids 824 to 1090 of TEK | AZD0530 |
| Amino acids 1125 to 1280 of NCOA2 | Lapatinib |
| Amino acids 480 to 729 of EXT1 | Nilotinib |
| Amino acids 149 to 248 of IKZF3 | Paclitaxel |
| Amino acids 17 to 268 of TSSK1B | Erlotinib |
| Amino acids 17 to 272 of TSSK1B | Erlotinib |
| Amino acids 190 to 442 of TNK2 | PD-0332991 |
| Amino acids 545 to 681 of SUZ12 | L-685458 |
| Amino acids 498 to 557 of GAB1 | PF2341066 |
| Amino acids 231 to 423 of EHBP1 | ZD-6474 |
| Amino acids 500 to 660 of CACNB2 | RAF265 |
| Amino acids 1256 to 1451 of NF1 | TAE684 |
| Amino acids 54 to 384 of GUCY2C | Irinotecan |
| Amino acids 76 to 288 of HDAC4 | Nilotinib |
| Amino acids 667 to 923 of PAPPA | AZD0530 |
| Amino acids 87 to 802 of MYH10 | AEW541 |
| Amino acids 642 to 955 of THRAP3 | Paclitaxel |
| Amino acids 400 to 502 of RASA1 | PHA-665752 |
| Amino acids 1780 to 2333 of ACACB | PLX4720 |
| Amino acids 295 to 515 of NEK5 | Paclitaxel |
| Amino acids 1075 to 1325 of MSH6 | RAF265 |
| Amino acids 408 to 731 of ADARB2 | AEW541 |
| Amino acids 408 to 731 of ADARB2 | Erlotinib |
| Amino acids 113 to 318 of DYRK1B | Erlotinib |
| Amino acids 266 to 598 of MINK1 | Erlotinib |
| Amino acids 213 to 377 of ZMYND10 | Lapatinib |
| Amino acids 161 to 372 of DYRK1A | Nutlin-3 |
| Amino acids 159 to 479 of DYRK1A | Nutlin-3 |
| Amino acids 124 to 398 of MLK4 | Nutlin-3 |
| Amino acids 125 to 397 of MLK4 | Nutlin-3 |
| Amino acids 1421 to 1848 of MYH10 | Nutlin-3 |
| Amino acids 23 to 94 of DTX1 | Paclitaxel |
| Amino acids 373 to 573 of RB1 | Panobinostat |
| Amino acids 82 to 249 of REM1 | PD-0325901 |
| Amino acids 56 to 166 of ERBB3 | PF2341066 |
| Amino acids 137 to 218 of PRKG1 | PF2341066 |
| Amino acids 96 to 299 of TEC | PF2341066 |
| Amino acids 533 to 842 of MSH3 | PHA-665752 |
| Amino acids 475 to 749 of FGFR3 | RAF265 |
| Amino acids 474 to 750 of FGFR3 | RAF265 |
| Amino acids 128 to 535 of CARS | Sorafenib |
| Amino acids 75 to 408 of GUCY2F | TKI258 |
| Amino acids 648 to 747 of SIRT1 | ZD-6474 |
| Amino acids 428 to 544 of SUZ12 | ZD-6474 |
| Amino acids 21 to 253 of URB2 | ZD-6474 |
| Amino acids 2497 to 2588 of WNK1 | ZD-6474 |

In some forms of the methods, the genetic feature in the drug-specific PFR is present in one or more cancer cells of the subject. In some forms of the methods, the subject is identified as having one or more cells having the genetic feature in the drug-specific PFR prior to treatment. In some forms of the methods, the genetic feature is detected in the drug-specific PFR in one or more cells of the subject prior to treatment.

In some forms of the methods, each genetic feature is either the presence of one or more genetic alterations or a lack of one or more genetic alterations.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows analysis at the functional region level allows us to gain novel insights from pharmacogenornics data. (a, b) Mapping of the different ERBB3 functions to specific regions of the protein. Each functional relationship can be associated to a specific domain or intrinsically disordered region in ERBB3. For example, physical interactions between ERBB3 and EGFR and NRG1 (line connecting EGFR and ERBB3 and NRG1 with ERBB3 in (a)) are mediated by EGF receptor domains (boxes 1 and 3 (from the left) on PFAM in (b)); effect of CDK5 on ERBB3 (arrow from CDK5 to ERBB3 in (a)) are mediated by the C- terminal intrinsically disordered regions (boxes 1, 2 and 3 (from the right) on IDR in (b); feedback of ERBB3 (arrow from and to ERBB3 in (a)) and physical interactions JAK3 with ERBB3 (line connecting JAK3 and ERBB3 in (a)) are mediated by the kinase domain (dark gray box on PFAM in (b)). (c) Methods focusing at the whole-protein level cannot find any association between ERBB3 mutations and the activity of PF2341066. (d) Mutations altering specifically the N-terminal EGF receptor are associated with lower drug activity. (e) Mutations affecting another PFR in ERBB3, its kinase domain (which mutations thus mainly affect other functional regions), are not associated with any changes in drug activity. (f) Venn diagram showing the different thresholds established in order to minimize false positives. PFRs were kept only when (I) p<0.001 when compared to cell lines with no mutation in the protein, (II) p<0.05 when compared to cell lines with mutations in other regions of the same protein, and (III) p>0.01 at the protein level.

FIG. 2 shows perturbations of different regions in the same protein can have different drug effects. Missense mutations in different PFRs of MSH6 lead to increased sensitivity towards three different drugs: AEW541, RAF265 and Lapatinib. The protein level analysis on the other hand reveals a potential association with Erlotinib. This highlights the complementarity between protein and PFR-centric approaches.

FIG. 3 shows validations of some predictions by e-Drug using complimentary datasets. Missense mutations in PIK3CA can have opposite effects in terms of AEW541 activity depending on the PFR affected. Mutations in the p85-binding and PIK accessory domains are associated with lower and higher drug activities respectively (upper panel). Integration of the analysis with proteomics data from TCPA led to a proposed mechanism for that result. It appears that IRS 1 protein expression is lower in cells with p85-binding mutations, but higher in those with PIK mutations (second panel). Moreover, Akt1 phosphorylation levels are higher in cell lines with p85-binding domain mutations (two lower panels).

FIG. 4 shows how PFR perturbations identified using data from cell lines predict the survival of patients treated with irinotecan. (a) Proteins with PFR associated to irinotecan resistance cannot be used to successfully strati& cancer patients treated with this drug, as there are no differences between patients with mutations in such proteins (broken line) and those without them. (solid) (b) Specific PFR in these proteins do predict the outcome of cancer patients. Patients with mutations altering the PFRs found using CCLE (rapidly falling line) have worse outcomes that those with mutations in other regions of the same protein (non-falling line) or no mutations (moderately falling line).

FIG. 5 is an enrichment map of the proteins associated with differential drug activity at both whole-protein and individual region levels. A gene-set enrichment analysis was performed by comparing Gene Ontology (GO) annotations of the 316 proteins associated with different drugs at both levels of resolution (whole-protein and individual PFRs) against the whole human genome. All the GO terms identified here showed an enrichment in the biomarker group, and most of them relate to pathways and functions associated with carcinogenesis, metastasis, and drug resistance, such as regulation of cell proliferation, kinase activity, cell migration, cell adhesion, MAPK cascade, or response to hypoxia. In the figure, GO terms are connected when they are related according to the gene ontology.

### DETAILED DESCRIPTION OF THE INVENTION

The disclosed method and compositions may be understood more readily by reference to the following detailed description of particular embodiments and the Example included therein and to the Figures and their previous and following description.

The general approach to correlating genetic alterations with drug effects assumes that mutations in a gene will have the same consequences regardless of their location. While this assumption might be correct in some cases, such an approach cannot fully deal with situations in which different mutations in the same protein have different effects depending on which region of the protein is being altered (Kobayashi et al., New England Journal of Medicine 325:7 (2005)). This idea can be easily visualized if one thinks about the modularity of proteins. For instance, a receptor tyrosine kinase, such as EGFR, usually has an extracellular region, which is responsible for the interaction with the ligand or with other receptors, and an intracellular kinase domain, which in turn is responsible for the phosphorylation of its substrates. A phenotype, such as the response towards a drug, can be influenced by alterations of these proteins at the whole-protein level (changes in expression, deletion of or epigenetic silencing of a gene), but also by mutations modifying only the extracellular or the kinase domains. More importantly, even though it is likely that each of the three types of alterations (whole-protein, only in the extracellular region or only in the kinase domain) will have different consequences (Sahni et al., Curr Opin Genet Dev 23:649-657 (2013)), only those involving the whole protein have been studied. This is evidence that altering different functional regions within the same protein can lead to dramatically distinct phenotypes.

Both the recognition of this problem and its solution are described here. By focusing on individual regions instead of whole proteins, correlations were identified that predict the activity of anticancer drugs. Proteomic data from both cancer cell lines and actual cancer patients was used to explore the molecular mechanisms underlying some of these region-drug associations. It is also demonstrated that associations found between protein regions and drugs using only data from cancer cell lines can predict the survival of cancer patients.

Disclosed are analyses that separate the effects of mutations in different protein functional regions (PFRs), including protein domains and intrinsically disordered regions (IDRs), on drug activity. Using this approach 171 new associations were identified between mutations in specific PFRs and changes in the activity of 24 drugs that couldn't be recovered by traditional gene-centric analyses. The results demonstrate how focusing on individual protein regions can provide new insights into the mechanisms underlying the drug sensitivity of cancer cell lines. Moreover, while these new correlations are identified using only data from cancer cell lines, some of the correlations have been validated using data from actual cancer patients. The discoveries described herein highlight how gene-centric experiments (such as systematic knock-out or silencing of individual genes) are missing relevant effects mediated by perturbations of specific protein regions. Some of the identified associations are described in Table 2 and others are available at the website cancer3d.org.

To determine how perturbations of specific PFRs influence the sensitivity of cancer cell lines towards specific drugs a new analysis protocol called e-Drug was developed. This protocol analyzes each functional region within a protein separately and finds those associated with changes in the activity of anticancer drugs. For the algorithm, the definition of PFRs includes protein domains and intrinsically disordered regions. In the demonstrations herein, the protein domains included both those present in Pfam database and those predicted to exist using domain analysis tools. Pfam protein domains have been used previously to study the molecular mechanisms underlying the pleiotropy of certain genes, especially those related to Mendelian disorders (Zhong et al., Mol Syst Biol 5:321 (2009); Wang et al., Nat Biotechnol 30:159-164 (2012)), and cancer (Ryan et al., Nat Rev Genet 14:865-879 (2013)); Porta-Pardo and Godzik, Bioinformatics doi:10.1093/bioinformatics/btu499 (2014)); Nehrt et al., Genomics 13 Suppl 4:S9 (2012)), but not cancer pharmacogentics (that is, correlation of protein-specific genetic alterations to drug activity). In the context of the analysis of drug-related data, PFRs have been used to study phenomena such as polypharmacology or the structural details underlying interactions between drugs and domains (Moya-Garcia and Ranea, Bioinformatics 29:1934-1937 (2013)); Kruger et al., Bioinformatics 13 Suppl 17:S11 (2012)), but not to study cancer pharmacogenomic datasets.

The disclosed methods generally involve assessing correlations between compounds, genetic features, diseases, and effects. The methods can use any source of data regarding the compounds, genetic features, diseases, and effects. The disclosed methods make use of statistical methods that are known and have been applied to find correlations in these types of data. Such methods are known and can be applied to the disclosed methods. In some forms of the disclosed methods, the correlations calculated involve specific sub-regions of proteins that have not been correlated to disease-associated effects of compounds. Although the subsets and subdivisions of data used for the disclosed correlations and methods are new, the basic techniques applied are well known. Known techniques for correlation analysis can be adapted for use with the disclosed methods. Similarly, known techniques for detection of genetic features in cells and subjects can be adapted for use in the disclosed methods. Data sets for use in the disclosed methods can be, for example, known data sets, publicly maintained and available data sets, proprietary data sets, newly generated data sets, and combinations thereof. An example of the disclosed methods was demonstrated using publicly available data sets combined with new data categories (PFRs) derived from the public data sets.

Unless the context clearly indicates otherwise, reference to correlations herein refer to statistically significant correlations (p<0.05). In some forms of the methods, hits can be defined more stringently, accepting only correlations at p<0.01. As described herein, this more stringent standard can be useful when working with small data sets.

Any suitable statistical method can be used to determine correlation. In statistical methods that use a different measure of statistical significance, correlation refers to the standard level of statistical significance for that method.

A drug-associated disease is a disease for which a compound is known to affect some instances of the disease.

A disease-associated compound is a compound that is known to affect some instances of the disease.

A genetic feature is any sequence, mutation, alteration, variant, allele, and the like that is specified by the genetic material of a cell. Where the cell is part of a multicellular organism, such as a subject, the genetic feature can be said to a genetic feature of the organism. A genetic alteration is a genetic feature where the sequence of the genetic material is altered from the wild type sequence, dominant allele sequence, or some other comparison sequence. In the context of proteins, a genetic feature is any sequence, mutation, alteration, variant, allele, and the like in the gene that encodes the protein. A protein-specific genetic feature is a genetic feature that specified a sequence, mutation, alteration, variant, allele, and the like of the protein. In the context of genes, a genetic feature is any sequence, mutation, alteration, variant, allele, and the like in the gene, including the introns, expression, and regulatory sequences. Genetic features can be defined by the presence or absence of a sequence, mutation, alteration, variant, allele, and the like. For example, a genetic feature can be the absence of a variant sequence.

An intrinsically disordered region (IDR) is a region of a protein that is intrinsically disordered. For example, a protein region that is disordered as indicated by Foldindex can be considered an intrinsically disordered region.

A protein functional region (PFR) is a domain or IDR of a protein. For example, a domain identified in Pfam and/or using a domain identifying algorithm such as AIDA can be considered a protein functional region. A PFR group is a combination of two or more, but fewer than all, of the PFRs in a protein. A whole protein is all of the protein. A whole protein includes, for example, all of the PFRs, functional domains, IDRs, PFR groups, etc. in the protein. A protein unit is a PFR, a PFR group, or a whole protein. Although the term protein functional domain (PFR) refers to domains and although the term protein domain has other meanings in the art, the terms PFR (protein functional domain) and protein unit are not intended to be limited to a classical definition of protein domains (although the disclosed methods can use and include classically defined protein domains as PFRs and protein units). Rather, protein functional domains can include any region, subsequence, or combination of regions, subsequences, or both that can be identified as having functional distinctness from other regions and subsequences in a protein. A phosphorylation site in a protein is an example of a region of a protein (perhaps a single amino acid) that is not a classical protein domain but that has a functional distinctness from other regions of the protein.

A set of PFRs is a collection or combination of two or more PFRs. The PFRs in a set of PFRs can come from the same protein, from different proteins, or a combination. A set of PFR groups is a collection or combination of two or more PFR groups. The PFR groups in a set of PFR groups can come from the same protein, from different proteins, or a combination. A set of whole proteins is a collection or combination of two or more whole proteins. A set of protein units is a collection or combination of two or more protein units. The protein units in a set of protein units can come from the same protein, from different proteins, or a combination. Any combination of protein units can be combined in a set of protein units. For example, a set of protein units can be made up of a set of PFRs, a set of PFR groups, a set of PFRs and PFR groups, a set of PFRs and whole proteins, a set of PFR groups and whole proteins, and a set of PFRs, PFR groups, and whole proteins. These sets can also specify any feature of the PFRs, PFR groups, protein units, or proteins in the set. For example, in a set of disease-associated protein units all of the protein units in the set are disease-associated protein units.

A drug-specific protein unit is a protein unit of a protein (that is less than the whole protein) where genetic features in the protein unit are correlated with an effect of a compound but where genetic features in the protein as a whole are not correlated with the effect of the compound. In such a case, the compound is a protein unit-specific compound for the protein unit, the protein unit is a drug-specific protein unit for the compound, and the effect of the compound that is correlated with genetic features in the protein unit is a protein unit-associated effect of the compound and for the protein unit.

A drug-specific PFR is a PFR of a protein where genetic features in the PFR are correlated with an effect of a compound but where genetic features in the protein as a whole are not correlated with the effect of the compound. In such a case, the compound is a PFR-specific compound for the PFR, the PFR is a drug-specific PFR for the compound, and the effect of the compound that is correlated with genetic features in the PFR is a PFR-associated effect of the compound and for the PFR. Drug-specific PFRs are not identified merely by the fact that a specific genetic feature in the PFR has been individually correlated with a drug or drug effect. Rather, it is the correlation of genetic features in the PFR in general with the drug or drug effect where there is no correlation of genetic features in the PFR-containing protein as a whole with the drug or drug effect. Similarly, A PFR is not a drug-specific PFR unless there is no correlation of genetic features in the PFR-containing protein as a whole with the drug or drug effect.

A drug-specific PFR group is a PFR group of a protein where genetic features in the PFR group are correlated with an effect of a compound but where genetic features in the protein as a whole are not correlated with the effect of the compound. In such a case, the compound is a PFR group-specific compound for the PFR group, the PFR group is a drug-specific PFR group for the compound, and the effect of the compound that is correlated with genetic features in the PFR group is a PFR group-associated effect of the compound and for the PFR group.

A drug-specific protein is a protein where genetic features in the protein as a whole are correlated with an effect of a compound. In such a case, the compound is a protein-specific compound for the protein, the protein is a drug-specific protein for the compound, and the effect of the compound that is correlated with genetic features in the protein is a protein-associated effect of the compound and for the protein.

A drug-specific set of protein units is a set of protein units of one or more proteins where genetic features in the set of protein units are correlated with an effect of a compound. In such a case, the compound is a protein unit-specific compound for the set of protein units, the set of protein units is a drug-specific set of protein units for the compound, and the effect of the compound that is correlated with genetic features in the set of protein units is a protein unit-associated effect of the compound and for the set of protein units.

In some cases, for one or more of the proteins from which one or more of the protein units in the set of protein units come, genetic features in each of the one or more proteins as a whole are not correlated with the effect of the compound. For example, for one of the proteins from which one or more of the protein units in the set of protein units come, genetic features in the one protein as a whole are not correlated with the effect of the compound. As another example, for all of the proteins from which the protein units in the set of protein units come, genetic features in each of the proteins as a whole are not correlated with the effect of the compound. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A PFR-specific compound is a compound where an effect of the compound is correlated with genetic features in a PFR of a protein but where the effect of the compound is not correlated with genetic features in the protein as a whole. In such a case, the PFR is a drug-specific PFR for the compound, the compound is PFR-specific compound for the PFR, and the effect of the compound that is correlated with genetic features in the PFR is a PFR-associated effect of the compound and for the PFR.

A PFR group-specific compound is a compound where an effect of the compound is correlated with genetic features in a PFR group of a protein but where the effect of the compound is not correlated with genetic features in the protein as a whole. In such a case, the PFR group is a drug-specific PFR group for the compound, the compound is PFR group-specific compound for the PFR group, and the effect of the compound that is correlated with genetic features in the PFR group is a PFR group-associated effect of the compound and for the PFR group.

A protein unit-specific compound is a compound where an effect of the compound is correlated with genetic features in a protein unit of a protein (that is less than the whole protein) but where the effect of the compound is not correlated with genetic features in the protein as a whole. In such a case, the protein unit is a drug-specific protein unit for the compound, the compound is protein unit-specific compound for the protein unit, and the effect of the compound that is correlated with genetic features in the protein unit is a protein unit-associated effect of the compound and for the protein unit.

A protein-specific compound is a compound where an effect of the compound is correlated with genetic features in a protein as a whole. In such a case, the protein is a drug-specific protein for the compound, the compound is protein-specific compound for the protein, and the effect of the compound that is correlated with genetic features in the protein is a protein-associated effect of the compound and for the protein.

A protein unit set-specific compound is a compound where an effect of the compound is correlated with genetic features in a set of protein units of one or more proteins. In such a case, the set of protein units is a drug-specific set of protein units for the compound, the compound is protein unit set-specific compound for the set of protein units, and the effect of the compound that is correlated with genetic features in the set of protein units is a protein unit set-associated effect of the compound and for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A PFR-associated effect is an effect of a compound that is correlated with genetic features in a PFR of a protein but where the effect of the compound is not correlated with genetic features in the protein as a whole. In such a case, the PFR is a drug-specific PFR for the compound, the compound is a PFR-specific compound for the PFR, and the effect is a PFR-associated effect of the PFR.

A PFR group-associated effect is an effect of a compound that is correlated with genetic features in a PFR group of a protein but where the effect of the compound is not correlated with genetic features in the protein as a whole. In such a case, the PFR group is a drug-specific PFR group for the compound, the compound is a PFR group-specific compound for the PFR group, and the effect is a PFR group-associated effect of the PFR group.

A protein unit-associated effect is an effect of a compound that is correlated with genetic features in a protein unit of a protein (that is less than the whole protein) but where the effect of the compound is not correlated with genetic features in the protein as a whole. In such a case, the protein unit is a drug-specific protein unit for the compound, the compound is a protein unit-specific compound for the protein unit, and the effect is a protein unit-associated effect of the protein unit.

A protein-associated effect is an effect of a compound that is correlated with genetic features in a protein as a whole. In such a case, the protein is a drug-specific protein for the compound, the compound is a protein-specific compound for the protein, and the effect is a protein-associated effect of the protein.

A protein unit set-associated effect is an effect of a compound that is correlated with genetic features in a set of protein units of one or more proteins. In such a case, the set of protein units is a drug-specific set of protein units for the compound, the compound is a protein unit set-specific compound for the set of protein units, and the effect is a protein unit set-associated effect of the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

An effect-associated PFR is a PFR of a protein where genetic features in the PFR are correlated with an effect of a compound but where genetic features in the protein as a whole are not correlated with the effect of the compound. In such a case, the effect is a PFR-associated effect of the PFR, the PFR is a drug-specific PFR for the compound, and the compound is a PFR-specific compound for the PFR.

An effect-associated PFR group is a PFR group of a protein where genetic features in the PFR group are correlated with an effect of a compound but where genetic features in the protein as a whole are not correlated with the effect of the compound. In such a case, the effect is a PFR group-associated effect of the PFR group, the PFR group is a drug-specific PFR group for the compound, and the compound is a PFR group-specific compound for the PFR group.

An effect-associated protein unit is a protein unit of a protein (that is less than the whole protein) where genetic features in the protein unit are correlated with an effect of a compound but where genetic features in the protein as a whole are not correlated with the effect of the compound. In such a case, the effect is a protein unit-associated effect of the protein unit, the protein unit is a drug-specific protein unit for the compound, and the compound is a protein unit-specific compound for the protein unit.

An effect-associated protein is a protein where genetic features in the protein as a whole are correlated with an effect of a compound. In such a case, the effect is a protein-associated effect of the protein, the protein is a drug-specific protein for the compound, and the compound is a protein-specific compound for the protein.

An effect-associated set of protein units is a set of protein units of one or more proteins where genetic features in the set of protein units are correlated with an effect of a compound. In such a case, the effect is a protein unit set-associated effect of the set of protein units, the set of protein units is a drug-specific set of protein units for the compound, and the compound is a protein unit set-specific compound for the set of protein unit. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A PFR/drug-specific genetic feature is a genetic feature in a PFR of a protein where genetic features in the PFR are correlated with an effect of a compound but where genetic features in the protein as a whole are not correlated with the effect of the compound. In such a case, the PFR is a genetic feature/drug-specific PFR for the genetic feature and a drug-specific PFR for the compound, and the compound is a PFR-specific compound for the PFR.

A PFR group/drug-specific genetic feature is a genetic feature in a PFR group of a protein where genetic features in the PFR group are correlated with an effect of a compound but where genetic features in the protein as a whole are not correlated with the effect of the compound. In such a case, the PFR group is a genetic feature/drug-specific PFR group for the genetic feature and a drug-specific PFR group for the compound, and the compound is a PFR group-specific compound for the PFR group.

A protein unit/drug-specific genetic feature is a genetic feature in a protein unit of a protein (that is less than the whole protein) where genetic features in the protein unit are correlated with an effect of a compound but where genetic features in the protein as a whole are not correlated with the effect of the compound. In such a case, the protein unit is a genetic feature/drug-specific protein unit for the genetic feature and a drug-specific protein unit for the compound, and the compound is a protein unit-specific compound for the protein unit.

A protein/drug-specific genetic feature is a genetic feature in a protein where genetic features in the protein as a whole are correlated with an effect of a compound. In such a case, the protein is a genetic feature/drug-specific protein for the genetic feature and a drug-specific protein for the compound, and the compound is a protein-specific compound for the protein.

A protein unit set/drug-specific genetic feature is a genetic feature in a set of protein units of one or more proteins where genetic features in the set of protein units are correlated with an effect of a compound. In such a case, the set of protein units is a genetic feature/drug-specific set of protein units for the genetic feature and a drug-specific set of protein units for the compound, and the compound is a protein unit set-specific compound for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A genetic feature /drug-specific PFR is a PFR of a protein where genetic features in the PFR are correlated with an effect of a compound but where genetic features in the protein as a whole are not correlated with the effect of the compound. In such a case, a genetic feature in the PFR is a PFR/drug-specific genetic feature, the PFR is a drug-specific PFR for the compound, and the compound is a PFR-specific compound for the PFR.

A genetic feature /drug-specific PFR group is a PFR group of a protein where genetic features in the PFR group are correlated with an effect of a compound but where genetic features in the protein as a whole are not correlated with the effect of the compound. In such a case, a genetic feature in the PFR group is a PFR group/drug-specific genetic feature, the PFR group is a drug-specific PFR group for the compound, and the compound is a PFR group-specific compound for the PFR group.

A genetic feature /drug-specific protein unit is a protein unit of a protein (that is less than the whole protein) where genetic features in the protein unit are correlated with an effect of a compound but where genetic features in the protein as a whole are not correlated with the effect of the compound. In such a case, a genetic feature in the protein unit is a protein unit/drug-specific genetic feature, the protein unit is a drug-specific protein unit for the compound, and the compound is a protein unit-specific compound for the protein unit.

A genetic feature /drug-specific protein is a protein where genetic features in the protein as a whole are correlated with an effect of a compound. In such a case, a genetic feature in the protein is a protein/drug-specific genetic feature, the protein is a drug-specific protein for the compound, and the compound is a protein-specific compound for the protein.

A genetic feature /drug-specific set of protein units is a set of protein units of one or more proteins where genetic features in the set of protein units are correlated with an effect of a compound. In such a case, a genetic feature in the set of protein units is a protein unit set/drug-specific genetic feature, the set of protein units is a drug-specific set of protein units for the compound, and the compound is a protein unit set-specific compound for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A disease-associated effect is an effect of a compound on at least some instances of a disease. In such a case, the disease is a drug-associated disease for the compound and the effect is an effect of the compound. An effect-associated disease is a disease for which a compound has an effect in at least some instances of the disease. In such a case, the disease is a drug-associated disease for the compound and the effect is an effect of the compound.

A PFR/disease-associated compound is a compound where the compound is a disease-associated compound for a disease, where an effect of the compound is correlated with genetic features in a PFR of a protein but where the effect of the compound is not correlated with genetic features in the protein as a whole, and where the effect is a disease-associated effect for the disease. In such a case, the effect is a PFR-associated effect of the compound and for the PFR, the disease is an effect-associated disease for the effect, the PFR is a drug-specific PFR for the compound, and the compound is PFR-specific compound for the PFR.

A PFR group/disease-associated compound is a compound where the compound is a disease-associated compound for a disease, where an effect of the compound is correlated with genetic features in a PFR group of a protein but where the effect of the compound is not correlated with genetic features in the protein as a whole, and where the effect is a disease-associated effect for the disease. In such a case, the effect is a PFR group-associated effect of the compound and for the PFR group, the disease is an effect-associated disease for the effect, the PFR group is a drug-specific PFR group for the compound, and the compound is PFR group-specific compound for the PFR group.

A protein unit/disease-associated compound is a compound where the compound is a disease-associated compound for a disease, where an effect of the compound is correlated with genetic features in a protein unit of a protein (that is less than the whole protein) but where the effect of the compound is not correlated with genetic features in the protein as a whole, and where the effect is a disease-associated effect for the disease. In such a case, the effect is a protein unit-associated effect of the compound and for the protein unit, the disease is an effect-associated disease for the effect, the protein unit is a drug-specific protein unit for the compound, and the compound is protein unit-specific compound for the protein unit.

A protein/disease-associated compound is a compound where the compound is a disease-associated compound for a disease, where an effect of the compound is correlated with genetic features in a protein as a whole and where the effect is a disease-associated effect for the disease. In such a case, the effect is a protein-associated effect of the compound and for the protein, the disease is an effect-associated disease for the effect, the protein is a drug-specific protein for the compound, and the compound is protein-specific compound for the protein.

A protein unit set/disease-associated compound is a compound where the compound is a disease-associated compound for a disease, where an effect of the compound is correlated with genetic features in a set of protein units of one or more proteins and where the effect is a disease-associated effect for the disease. In such a case, the effect is a protein unit set-associated effect of the compound and for the set of protein units, the disease is an effect-associated disease for the effect, the set of protein units is a drug-specific set of protein units for the compound, and the compound is protein unit set-specific compound for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A PFR-associated disease is a disease where an effect of a disease-associated compound for the disease is correlated with genetic features in a PFR of a protein but where the effect of the compound is not correlated with genetic features in the protein as a whole and where the effect is a disease-associated effect for the disease. In such a case, the effect is a PFR-associated effect of the compound and for the PFR, the disease is an effect-associated disease for the effect, the PFR is a drug-specific PFR for the compound, and the compound is PFR-specific compound for the PFR.

A PFR group-associated disease is a disease where an effect of a disease-associated compound for the disease is correlated with genetic features in a PFR group of a protein but where the effect of the compound is not correlated with genetic features in the protein as a whole and where the effect is a disease-associated effect for the disease. In such a case, the effect is a PFR group-associated effect of the compound and for the PFR group, the disease is an effect-associated disease for the effect, the PFR group is a drug-specific PFR group for the compound, and the compound is PFR group-specific compound for the PFR group.

A protein unit-associated disease is a disease where an effect of a disease-associated compound for the disease is correlated with genetic features in a protein unit of a protein (that is less than the whole protein) but where the effect of the compound is not correlated with genetic features in the protein as a whole and where the effect is a disease-associated effect for the disease. In such a case, the effect is a protein unit-associated effect of the compound and for the protein unit, the disease is an effect-associated disease for the effect, the protein unit is a drug-specific protein unit for the compound, and the compound is protein unit-specific compound for the protein unit.

A protein-associated disease is a disease where an effect of a disease-associated compound for the disease is correlated with genetic features in a protein as a whole and where the effect is a disease-associated effect for the disease. In such a case, the effect is a protein-associated effect of the compound and for the protein, the disease is an effect-associated disease for the effect, the protein is a drug-specific protein for the compound, and the compound is protein-specific compound for the protein.

A protein unit set-associated disease is a disease where an effect of a disease-associated compound for the disease is correlated with genetic features in a set of protein units of one or more proteins and where the effect is a disease-associated effect for the disease. In such a case, the effect is a protein unit set-associated effect of the compound and for the set of protein units, the disease is an effect-associated disease for the effect, the set of protein units is a drug-specific set of protein units for the compound, and the compound is protein unit set-specific compound for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

A disease-associated PFR is a PFR of a protein where genetic features in the PFR are correlated with an effect of a disease-associated compound for the disease but where genetic features in the protein as a whole are not correlated with the effect of the compound and where the effect is a disease-associated effect for the disease. In such a case, the effect is a PFR-associated effect of the compound and for the PFR, the disease is an effect-associated disease for the effect, the PFR is a drug-specific PFR for the compound, and the compound is PFR-specific compound for the PFR.

A disease-associated PFR group is a PFR group of a protein where genetic features in the PFR group are correlated with an effect of a disease-associated compound for the disease but where genetic features in the protein as a whole are not correlated with the effect of the compound and where the effect is a disease-associated effect for the disease. In such a case, the effect is a PFR group-associated effect of the compound and for the PFR group, the disease is an effect-associated disease for the effect, the PFR group is a drug-specific PFR group for the compound, and the compound is PFR group-specific compound for the PFR group.

A disease-associated protein unit is a protein unit of a protein (that is less than the whole protein) where genetic features in the protein unit are correlated with an effect of a disease-associated compound for the disease but where genetic features in the protein as a whole are not correlated with the effect of the compound and where the effect is a disease-associated effect for the disease. In such a case, the effect is a protein unit-associated effect of the compound and for the protein unit, the disease is an effect-associated disease for the effect, the protein unit is a drug-specific protein unit for the compound, and the compound is protein unit-specific compound for the protein unit.

A disease-associated protein is a protein where genetic features in the protein as a whole are correlated with an effect of a disease-associated compound for the disease and where the effect is a disease-associated effect for the disease. In such a case, the effect is a protein-associated effect of the compound and for the protein, the disease is an effect-associated disease for the effect, the protein is a drug-specific protein for the compound, and the compound is protein-specific compound for the protein.

A disease-associated set of protein units is a set of protein units of one or more proteins where genetic features in the set of protein units are correlated with an effect of a disease-associated compound for the disease and where the effect is a disease-associated effect for the disease. In such a case, the effect is a protein unit set-associated effect of the compound and for the set of protein units, the disease is an effect-associated disease for the effect, the set of protein units is a drug-specific set of protein units for the compound, and the compound is protein unit set-specific compound for the set of protein units. This applies to any set of protein units, including, for example, a set of PFRs, a set of PFR groups, a set of PFRs and PFR groups, a set of PFRs and whole proteins, a set of PFR groups and whole proteins, and a set of PFRs, PFR groups, and whole proteins.

In some forms of the methods, at least one of the protein units in the set of drug-specific protein units is a PFR or a PFR group of a protein, where genetic features in the PFR or PFR group of the protein are correlated with an effect of the compound but where genetic features in the protein as a whole are not correlated with the effect of the compound. In some forms of the methods, one or more of the protein units in the set of drug-specific protein units is a PFR or a PFR group of a protein, where genetic features in the PFR or PFR group of the protein are correlated with an effect of the compound but where genetic features in the protein as a whole are not correlated with the effect of the compound.

In some forms of the methods, at least one of the protein units in the set of drug-specific protein units is a PFR or a PFR group of a protein, where genetic features in the PFR or PFR group of the protein are correlated with an effect of the compound but where genetic features in the other PFRs or PFR groups of the protein are not correlated with the effect of the compound. In some forms of the methods, one or more of the protein units in the set of drug-specific protein units is a PFR or a PFR group of a protein, where genetic features in the PFR or PFR group of the protein are correlated with an effect of the compound but where genetic features in the other PFRs or PFR groups of the protein are not correlated with the effect of the compound.

In some forms of the methods, at least one of the protein units in the set of drug-specific protein units is a PFR or a PFR group of a protein, where genetic features in the PFR or PFR group of the protein are correlated with an effect of the compound but where genetic features in both the other PFRs or PFR groups of the protein and the protein as a whole are not correlated with the effect of the compound. In some forms of the methods, one or more of the protein units in the set of drug-specific protein units is a PFR or a PFR group of a protein, where genetic features in the PFR or PFR group of the protein are correlated with an effect of the compound but where genetic features in both the other PFRs or PFR groups of the protein and the protein as a whole are not correlated with the effect of the compound.

A disease-related cell is a type of cell of which some genetic features are correlated with a disease. For example, cancer cells are disease-related cells for cancer. Generally, disease-related cells are cells involved in and/or affected by the disease. But genetic features can be present in non-involved cells (such as when a subject's cells contain a disease-predisposing genetic feature). For some diseases, most or all of the cells of a subject can be disease-related cells. For example, genetic features correlated with sickle cell anemia are usually present in all of the cells of a subject with sickle cell anemia, including germline cells. Some cancer-related genes can have genetic features correlated with cancer or anticancer drug effects that are present in most or all of the cells of a subject (e.g., predisposing genetic features) and so most or all of the cells of the subject can be disease-related cells for genetic features in the cancer-related gene. Other genetic features correlated with cancer or anticancer drug effects will be found only in cancer cells and so only the cancer cells are disease-related cells for these genetic features. In the context of the disclosed methods, a disease-related cell is a cell of which some genetic features are or are expected to be PFR/disease-, PFR group/disease-, protein unit/disease-, and/or protein/disease-associated genetic features for the disease of interest.

A compound, including test compounds, can be any chemical, such as an inorganic chemical, an organic chemical, a protein, a peptide, a carbohydrate, a lipid, or a combination thereof. For use in the disclosed methods, the compound generally can be compounds with known or expected effects, such as therapeutic effects, on a disease, disorder, or condition. For test compounds, various predetermined concentrations of the compounds can be used for screening, such as 0.01 micromolar, 1 micromolar and 10 micromolar. Test compound controls can include the measurement of an effect in the absence of the test compound or comparison to a compound known to have the effect.

An effect can be any effect of a compound on a disease, disorder, condition, subject, or cell. For the disclosed methods, it is preferred that the effect be an effect that is relevant to a disease, condition, or disorder. A disease-associated effect is an effect of a compound on at least some instances of a disease. An effect on a disease is an effect on the course, symptoms, prognosis, terms, severity, etc. of the disease or an effect on cells that is or is expected to be relevant to affecting the course, symptoms, prognosis, terms, severity, etc. of the disease. Useful or desired effects for compounds to treat a disease are known and such effects are useful for the disclosed methods.

For both generation and supplementation of data sets involving genetic features and identification of subjects having disease- and drug-associated genetic features, relevant genetic features can be detected and identified using any appropriate samples. For example, genetic features can be identified in relevant biological, organ, tissue, fluid, or cell samples. The type of technique used to detect and identify genetic features can be selected based on, or can influence, which type of sample is used. For example, some techniques can use samples including a relatively large number of cells, some techniques can use a single cell, and others fall in between. Generally, the sample will include or be made up of disease-related cells. A cell can be in vitro. Alternatively, a cell can be in vivo and can be found in a subject.

A subject said to “have” a genetic feature means that one or more cells of the subject have the genetic feature. As discussed elsewhere herein, some, many, or all of a subject's cells may have a genetic feature, depending on the nature of the genetic feature and its relationship to the disease under examination. This is analogous to saying a subject has cancer when only some of the subject's cells are cancer cells. Generally, in the context of the disclosed methods, a subject having a genetic feature will have that genetic feature in one or more disease-related cells.

The disclosed methods can be used with and applied to any disease or condition. The disclosed methods allow identification and use of many more genetic features and so can be used to correlate these genetic features to diseases and conditions and to the effects of drugs and compounds to treat disease and conditions. Most disease and conditions are caused or affected by genetic features, and the effectiveness of many drugs and therapies are also affected by genetic features. The correlations assessed by the disclosed methods allow better identification and matching of disease, subject, and treatment.

In some forms of the methods, the disease can be cancer. The disease can be any cancer, including, for example, melanoma, non-Hodgkin's lymphoma, Hodgkin's disease, leukemias, plasmocytomas, sarcomas, adenomas, gliomas, thymomas, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers.

In some forms of the methods, the disease can be a disease of, for example, the heart, kidney, ureter, bladder, urethra, liver, prostate, heart, blood vessels, bone marrow, skeletal muscle, smooth muscle, various specific regions of the brain (including, but not limited to the amygdala, caudatenucleus, cerebellum, corpuscallosum, fetal, hypothalamus, thalamus), spinal cord, peripheral nerves, retina, nose, trachea, lungs, mouth, salivary gland, esophagus, stomach, small intestines, large intestines, hypothalamus, pituitary, thyroid, pancreas, adrenal glands, ovaries, oviducts, uterus, placenta, vagina, mammary glands, testes, seminal vesicles, penis, lymph nodes, thymus, and spleen. In some forms of the methods, the disease can be a cardiovascular disease, a neurological disease, a metabolic disease, a respiratory disease, or an autoimmune disease.

In some forms of the methods, the disease can be an autoimmune disease such as, but not limited to, rheumatoid arthritis, multiple sclerosis, insulin dependent diabetes, Addison's disease, celiac disease, chronic fatigue syndrome, inflammatory bowel disease, ulcerative colitis, Crohn's disease, Fibromyalgia, systemic lupus erythematosus, psoriasis, Sjogren's syndrome, hyperthyroidism/Graves disease, hypothyroidism/Hashimoto's disease, Insulin-dependent diabetes (type 1), Myasthenia Gravis, endometriosis, scleroderma, pernicious anemia, Goodpasture syndrome, Wegener's disease, glomerulonephritis, aplastic anemia, paroxysmal nocturnal hemoglobinuria, myelodysplastic syndrome, idiopathic thrombocytopenic purpura, autoimmune hemolytic anemia, Evan's syndrome, Factor VIII inhibitor syndrome, systemic vasculitis, dermatomyositis, polymyositis and rheumatic fever.

In some forms of the methods, the disease can be an infection with any of a variety of infectious organisms, such as viruses, bacteria, parasites and fungi. Infectious organisms can include, for example, viruses, (e.g., RNA viruses, DNA viruses, human immunodeficiency virus (HIV), hepatitis A, B, and C virus, herpes simplex virus (HSV), cytomegalovirus (CMV) Epstein-Barr virus (EBV), human papilloma virus (HPV)), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions.

As will be recognized, the disclosed methods can be used to assess correlation, identify subjects and compound, and treat virtually any disease, disorder, or condition where genetic features are involved in the disease.

As noted elsewhere herein, the disclosed methods generally involve assessing correlations between compounds, genetic features, diseases, and effects. The methods can use any source of data regarding the compounds, genetic features, diseases, and effects. The disclosed methods make use of statistical methods that are known and have been applied to find correlations in these types of data. Such methods are known and can be applied to the disclosed methods. In some forms of the disclosed methods, the correlations calculated involve specific sub-regions of proteins that have not been correlated to disease-associated effects of compounds. Although the subsets and subdivisions of data used for the disclosed correlations and methods are new, the basic techniques applied are well known. Known techniques for correlation analysis can be adapted for use with the disclosed methods. Similarly, known techniques for detection of genetic features in cells and subjects can be adapted for use in the disclosed methods. Data sets for use in the disclosed methods can be, for example, known data sets, publicly maintained and available data sets, proprietary data sets, newly generated data sets, and combinations thereof. An example of the disclosed methods was demonstrated using publicly available data sets combined with new data categories (PFRs) derived from the public data sets.

In some forms of the disclosed methods, drug-specific and disease-associated protein units are identified. This can be accomplished by, for example, assessing correlation between genetic features in a test set of protein units and the effect of a compound on a disease, where identification of a correlation between genetic features in the test set of protein units and the effect of the compound on a disease identify the test set of protein units as a drug-specific set of protein units for the compound and for the disease and identify the compound as a protein unit/disease-associated compound for the disease and for the test set of protein units. In some forms of the disclosed methods, disease-associated and protein unit-specific compounds are identified. This can be accomplished by, for example, assessing correlation between genetic features in a set of protein units and the effect of a test compound on a disease, where identification of a correlation between genetic features in the set of protein units and the effect of the test compound on a disease identify the test compound as a protein unit-specific compound for the set of protein units and for the disease and identify the set of protein units as a drug-specific set of protein units for the disease and for the test compound.

In some forms of the methods, identification of the correlations can be accomplished by identifying protein units in proteins, categorizing genetic features by protein unit, where the genetic features are present or not present in disease-related cells, categorizing the genetic features by whether the compound has the effect on the disease in subjects having the disease and having the genetic features or by whether the compound has the effect on the disease-related cells affected by the disease and having the genetic features, and calculating the level of correlation between genetic features in the protein units and the effect of the compound.

Identification of protein units can be accomplished by, for example, identifying functional domains and IDRs of proteins. Protein domains can be defined in any suitable manner. For example, classically defined protein domains are sections of a protein that have a distinct function or structural character from other or flanking sections of the protein. For example, ligand binding domain, transmembrane domain, intracellular domain, signaling domain. Numerous algorithms and tools exist for identifying protein domains based other sequence and other features. For example, protein domains can be annotated Pfam domains available from ENSEMBL. Pfam is a large collection of protein families, each represented by multiple sequence alignments and hidden Markov models (HMMs) (Internet site pfam.sanger.ac.uk/). Protein domains can also be identified using other tools, such as AIDA (ab initio domain assembly; Xu et al., Nucleic Acids Research 12:W308-W313 (2014) (Web Server issue); Internet site ffas.burnham.org/AIDA/), an algorithm based on remote homology. Protein domains identified in different ways can be combined and used together in the disclosed methods. Other databases of, and tools useful for identifying, protein domains include InterProScan, which is an integrated search in PROSITE, Pfam, PRINTS and other family and domain databases; InterPro is a database of protein families, domains and functional sites in which identifiable features found in known proteins can be applied to unknown protein sequences (web site ebi.ac.uk/Tools/pfa/iprscan/); CDD Search, which is a Conserved Domain Database Search @ NCBI (web site ncbi.nlm.nih.gov/Structure/cdd/wrpsb.cgi); PANTHER Families, which contains 6594 protein families, each with a phylogenetic tree relating modern-day genes in 48 organisms; expert biologists have divided each family into subfamilies, which are generally orthologous groups but may also contain recently duplicated paralogs; each family and subfamily is also represented as a hidden Markov model (HMM), which can be used to classify new sequences to an existing subfamily (web site pantherdb.org/panther/); TIGRFAMs are protein families based on Hidden Markov Models or HMMs; TIGRFAMs is a resource consisting of curated multiple sequence alignments, Hidden Markov Models (HMMs) for protein sequence classification, and associated information designed to support automated annotation of (mostly prokaryotic) proteins (web site tigr.org/TIGRFAMs/index.shtml); ProDom is a comprehensive set of protein domain families automatically generated from the SWISS-PROT and TrEMBL sequence databases (Internet site prodom.prabi.fr/prodom/current/html/home.php); DOUTfinder identifies sub-significant domain hits missed by other databases have failed (Internet site mendel.imp.ac.at/dout/); SYSTERS (short for SYSTEmatic Re-Searching) is a collection of graph-based algorithms to hierarchically partition a large set of protein sequences into homologous families and superfamilies; the methods are based on an all-against-all database search (using Smith-Waterman comparisons on a GeneMatcher machine) (Internet site systers.molgen.mpg.de/); The Conserved Domain Architecture Retrieval Tool (CDART) performs similarity searches of the NCBI Entrez Protein Database based on domain architecture, defined as the sequential order of conserved domains in proteins (web site ncbi.nlm.nih.gov/Structure/lexington/lexington.cgi?cmd=rps); PANDIT is a collection of multiple sequence alignments and phylogenetic trees covering many common protein domains (web site ebi.ac.uk/goldman-srv/pandit/); AnDom helps to assign structural domains to protein sequences and to classify them according to SCOP (Internet site coot.embl.de/AnDom/Usage.html); SUPERFAMILY is a database of structural and functional protein annotations for all completely sequenced organisms (Internet site supfam.mrc-lmb.cam.ac.uk/SUPERFAMILY/); ProtMap clusters proteins from complete genomes by sequence similarity into groups - COGs, or in case of viruses, VOGs; Genome ProtMap maps each protein from a COG/VOG back to its genome, and displays all the genomic segments coding for members of this particular group of related proteins (web site ncbi.nlm.nih.gov/sutils/protmap.cgi?cluster=COG4690E&result=map); ProtClustDB, the NCBI Entrez Protein Clusters database, is a collection of Reference Sequence (RefSeq) proteins from the complete genomes of prokaryotes, plasmids, and organelles grouped and annotated based on sequence similarity and protein function (web site ncbi.nlm.nih.gov/sites/entrez?db=proteinclusters); PROSITE consists of documentation entries describing protein domains, families and functional sites as well as associated patterns and profiles to identify them (web site expasy.ch/prosite/); ScanProsite scans a sequence against PROSITE or a pattern against the UniProt Knowledgebase (Swiss-Prot and TrEMBL) (web site expasy.ch/tools/scanprosite/); High-quality Automated and Manual Annotation of microbial Proteomes (HAMAP) is a system, based on manual protein annotation, that identifies and semi-automatically annotates proteins that are part of well-conserved families or subfamilies: the HAMAP families (web site expasy.ch/sprot/hamap/); SVM-Prot is web-based support vector machine software for functional classification of a protein from its primary sequence (Internet site jing.cz3.nus.edu.sg/cgi-bin/symprot.cgi); The PIRSF classification system is based on whole proteins rather than on the component domains; therefore, it allows annotation of generic biochemical and specific biological functions, as well as classification of proteins without well-defined domains (Internet site pir.georgetown.edu/pirsf/); CDTree is a protein domain hierarchy viewer and editor (web site ncbi.nlm.nih.gov/Structure/cdtree/cdtree.shtml); EVEREST is an automatic identification and classification of protein domains and combines methodologies from the fields of finite metric spaces, machine learning and statistical modeling and achieves state of the art results (web site everest.cs.huji.ac.il/index.php); ProtoNet provides automatic hierarchical classification of protein sequences; the site allows users to study the clustering as well as its qualities (web site protonet.cs.huji.ac.il/index.php); Pandora is a keyword-based analysis of protein sets by integration of annotation sources (web site pandora.cs.huji.ac.il/); Jevtrace is a implementation of the evolutionary trace method; the software expands on the evolutionary trace by allowing manipulation of the input data and parameters of analysis, and presents a number of novel tree inspired analysis of protein families (Internet site compbio.berkeley.edu/people/marcin/jevtrace/); SBASE is a collection of protein domain sequences collected from the literature, from protein sequence databases and from genomic databases (Vlahovicek et al., Nucleic Acids Res. 30(1):273-5 (2002));

the protein domains are defined by their sequence boundaries given by the publishing authors or in one of the primary sequence databases (Swiss-Prot, PIR, TREMBL etc.) (Internet site hydra.icgeb.trieste.it/sbase/); mkdom 2 is the program used to build the ProDom database (Internet site prodom.prabi.fr/prodom/xdom/welcome.html); The CluSTr database offers an automatic classification of UniProt Knowledgebase and IPI proteins into groups of related proteins; the clustering is based on analysis of all pairwise comparisons between protein sequences (web site ebi.ac.uk/clustr/).

Intrinsically disordered regions (IDRs) can be identified using any suitable technique. For example, Foldlndex (Prilusky et al., Bioinformatics 21(16): 3435-8 (2005)), which predicts regions that have a low hydrophobicity and high net charge (either loops or unstructured regions) and is based on charge/hydrophaty analyzed locally using a sliding window can be used. Other useful predicators of intrinsically disordered regions include charge/hydropathy method (Uversky et al., Proteins 41(3): 415-27 (2000)), which predicts fully unstructured domains (random coils), and is based on global sequence composition; CSpritz (Walsh et al., Nucleic Acids Res. 39:W190-6 (2011) (Web Server issue)), which predicts disorder definitions include: missing x-ray atoms (short) and DisProt style disorder (long); DisEMBL (Linding et al., Structure 11(11):1453-9 (2003)), which predicts LOOPS (regions devoid of regular secondary structure), HOT LOOPS (highly mobile loops), and REMARK465 (regions lacking electron density in crystal structure), and is based on neural networks trained on X-ray structure data; Disopred2 (Ward et al., J. Mol. Biol. 337(3): 635-45 (2004)), which predicts regions devoid of ordered regular secondary structure, and is based on cascaded support vector machine classifiers trained on PSI-BLAST profiles; ESpritz (Baldi et al., J. Mach. Learn. 4:575-602 (2003)), which predicts disorder definitions include: missing x-ray atoms (short), Disprot style disorder (long), and NMR flexibility, and is based on bi-directional neural networks with diverse and high quality data derived from the Protein Data Bank and DisProt; GeneSilico Metadisorder (Kozlowski et al., BMC Bioinformatics 13:111 (2012)), which predicts regions that lack a well-defined 3D structure under native conditions (REMARK-465); this is a meta method, which uses other disorder predictors and calculates a consensus optimized using ANN, filtering and other techniques; GlobPlot (Linding et al., Nucleic Acids Res. 31(13):3701-8 (2003)), which predicts regions with high propensity for globularity on the Russell/Linding scale (propensities for secondary structures and random coils), and is based on Russell/Linding scale of disorder; HCA (Hydrophobic Cluster Analysis; Faure and Callebaut, Bioinformatics doi: 10.1093/bioinformatics/btt271 (2013); website impmc.upmc.fr/˜callebau/HCA.html), which predicts hydrophobic clusters, which tend to form secondary structure elements, and is based on helical visualization of amino acid sequence; IUPforest-L (Han et al., BMC Bioinformatics 10:8 (2009)), which predicts long disordered regions in a set of proteins, Moreau-Broto auto-correlation function of amino acid indices (AAIs); IUPred (Dosztanyi et al., Bioinformatics 21(16):3433-4 (2005)), which predicts regions that lack a well-defined 3D-structure under native conditions, and is based on energy resulting from inter-residue interactions, estimated from local amino acid composition; MD (Meta-Disorder predictor; Schlessinger et al., PLoS ONE 4(2): e4433 (2009)), which predicts regions of different types (for example, unstructured loops and regions containing few stable intra-chain contacts); this is a neural-network based meta-predictor that uses different sources of information predominantly obtained from orthogonal approaches; MeDor (Metaserver of Disorder; Lieutaud et al., BMC Genomics 9(Suppl 2):S25 (2008)), which predicts regions of different types; MeDor provides a unified view of multiple disorder predictors; this is a meta method, which uses other disorder predictors (like Foldlndex, DisEMBL REMARK465, IUPred, RONN, etc.) and provides additional features (like HCA plot, Secondary Structure prediction, Transmembrane domains, etc.) that all together help the user in defining regions involved in disorder; MFDp (Mizianty et al., Bioinformatics 26(18): i489-96 (2010)), which predicts different types of disorder including random coils, unstructured regions, molten globules, and REMARK-465-based regions; this is an ensemble of 3 SVMs specialized for the prediction of short, long and generic disordered regions, which combines three complementary disorder predictors, sequence, sequence profiles, predicted secondary structure, solvent accessibility, backbone dihedral torsion angles, residue flexibility and B-factors; NORSp (Liu and Rost, Nucleic Acids Res. 31(13):3833-5 (2003)), which predicts regions with No Ordered Regular Secondary Structure (NORS), and is based on secondary structure and solvent accessibility; OnD-CRF (Wang and Sauer, Bioinformatics 24(11): 1401-2 (2008)), which predicts the transition between structurally ordered and mobile or disordered amino acids intervals under native conditions; OnD-CRF applies Conditional Random Fields, CRFs, which rely on features generated from the amino acid sequence and from secondary structure prediction; PONDR (Romero et al., Proteins 42(1):38-48 (2001); Xue et al., Biochim Biophys Acta. 1804(4):996-1010 (2010)), which predicts all regions that are not rigid including random coils, partially unstructured regions, and molten globules, and is based on local amino acid composition, flexibility, hydropathy, etc.; PreLink (Quevillon-Cheruel et al., Curr. Protein Pept. Sci. 8(2):151-60 (2007)), which predicts regions that are expected to be unstructured in all conditions, regardless of the presence of a binding partner, Compositional bias and low hydrophobic cluster content; RONN (Yang et al., Bioinformatics 21(16):3369-76 (2005)), which predicts regions that lack a well-defined 3D structure under native conditions, and is based on bio-basis function neural network trained on disordered proteins; SEG (Wootton, Comput Chem. 18(3):269-85 (1994)), which predicts low-complexity segments that is, “simple sequences” or “compositionally biased regions.” and is based on locally optimized low-complexity segments are produced at defined levels of stringency and then refined according to the equations of Wootton and Federhen; SPINE-D (Zhang et al., Journal of Biomolecular Structure and Dynamics 29(4):799-813 (2012)), which predicts output long/short disorder and semi-disorder (0.4-0.7) and full disorder (0.7-1.0); semi-disorder is semi-collapsed with some secondary structure; this is a neural network based three-state predictor based on both local and global features.

Categorizing genetic features by protein unit can be accomplished by, for example, determining or noting that the genetic feature falls within or overlaps with the protein unit or by determining or noting that a protein unit encompasses or overlaps with a genetic feature. Categorizing genetic features by whether a compound has an effect on a disease can be accomplished by, for example, determining or noting that the compound has the effect on the disease in subjects having the genetic feature in disease-related cells or determining or noting that the compound has the effect in disease-related cells having the genetic feature. Calculating the level of correlation between genetic features in protein units and the effect of a compound on a disease can be accomplished using any suitable statistical methods. Such methods are known and can be applied to the disclosed methods. In some forms of the disclosed methods, the correlations calculated involve specific sub-regions of proteins that have not been correlated to disease-associated effects of compounds. Although the subsets and subdivisions of data used for the disclosed correlations and methods are new, the basic techniques applied are well known. Known techniques for correlation analysis can be adapted for use with the disclosed methods.

In some forms, the disclosed methods look for protein units that, when mutated, correlate with an effect of the different test compounds. Subjects (or cells) can be divided into those that have a genetic feature (e.g., mutation) in the protein unit being studied and those that do not. A Wilcoxon test, for example, can then be performed comparing the level of effect of each test compound in the two groups and keeping those with a p-value below, for example, 0.01. Finally, for those protein units associated to a certain test compound, the level of effect of that test compound on the subjects (or cells) having genetic features in the protein unit can be compared to the level of effect of that test compound on the subjects (or cells) having genetic features in other regions of the gene. By doing this, protein units that are significantly different from the rest of the gene can be identified. In cases where the number of subjects or cells in both groups is lower and where fewer tests are performed, a significance threshold of 0.05 instead of 0.01 can be used. In some forms of the methods, true positives can be considered those protein units that passed both thresholds and that are not in proteins that show an association (p<0.01) with the same compound at the whole-protein level. In some forms of the methods, the analysis can performed independently for each protein unit. In the case that a protein contains two overlapping protein units, the analysis can be performed on each one of them independently, returning their corresponding results. In other forms of the method, the analysis can performed together for all of the protein units in a set of protein units. For example, the subjects or cells having a genetic feature in all of the protein units in the set of protein units are one category and subjects or cells that do not have a genetic feature in all of the protein units in the set are in the other category.

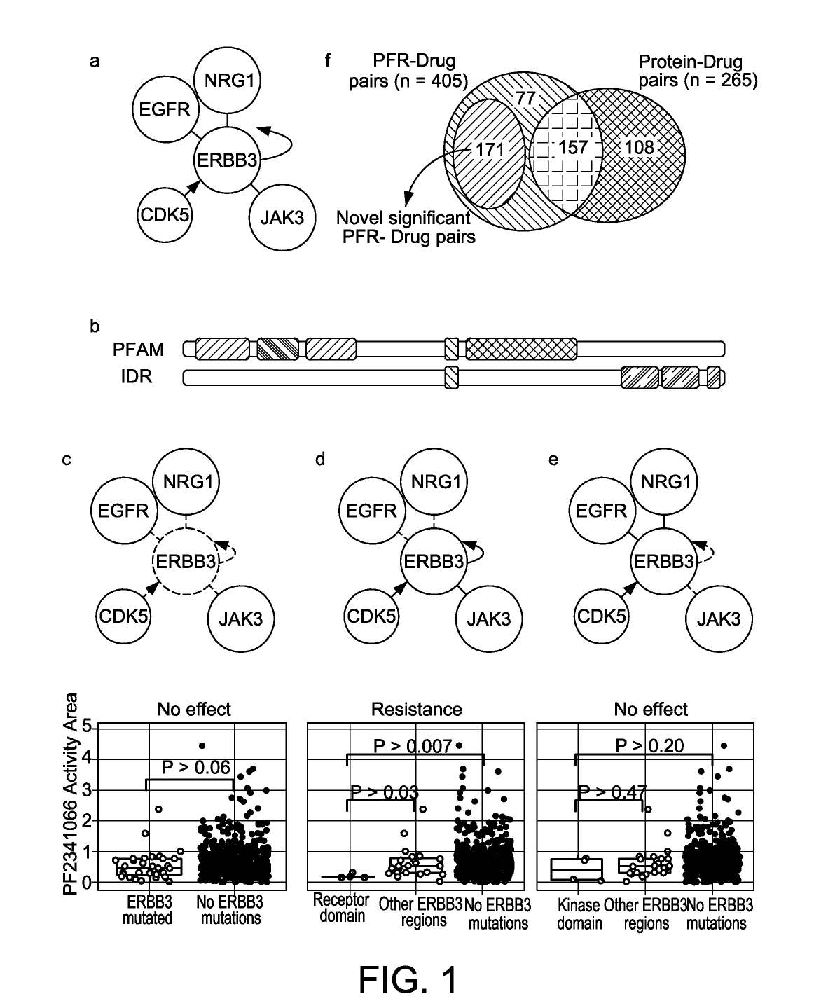

One of the problems that arise when analyzing protein units instead of whole proteins is that the statistical power of the sample decreases, as there are fewer subjects or cells with genetic features in the individual regions and the number of correlations being tested increases, making multiple-testing corrections more stringent. To overcome these limitations and decrease the number of false positives among the associations, different thresholds can be used for an association to be considered positive (see, e.g., FIG. 1). For example, the p-value of comparing the effect of compounds between subjects or cells with mutations in the protein unit against those without them generally can be below 0.01. The analysis can then be repeated at the protein level and all the pairs that are also identified there (p<0.01) can be removed. Then, for the remaining pairs, the effect of the compound on the subjects or cells can be compared with genetic features in the protein unit against subjects or cells with genetic features in other regions of the same protein.

The disclosed methods can be used to identify subjects that have or lack one or more genetic features that are correlated with a disease, compound, compound effect, etc. Thus, the disclosed methods can be used to, for example, stratify a population of subjects based on the presence or absence of one or more genetic features. In one important form, populations of subjects can be stratified into those that should be treated with a given compound and those that should not, based on the presence or absence of one or more genetic features correlated with an effect of the compound on the relevant disease. The subject population can be any group, set, or collection of subjects. Generally, subject populations for use with the disclosed methods can be populations of subjects that have or at risk for a relevant disease. In other forms of the method, a subject population can be stratified both by the presence or absence of a disease and by the presence or absence of one or more genetic features.

Stratification of subject populations is useful, for example, because it can contribute to improving the effectiveness of a treatment of a disease in a population of subjects that have the disease. In a simple form, effectiveness of treatment of the subject population is improved by treating a subject having genetic features in a drug-specific set of protein units in one or more disease-related cells with a protein unit-specific compound for the set of protein units and for the disease and refraining from treating a subject that does not have genetic features in one or more members of the drug-specific set of protein units of one or more disease-related cells with the protein unit-specific compound. This is a goal of personalized medicine that the disclosed methods can advance.

Different PFRs and protein units can have similar, different, or synergistic relationships to drug effects and diseases. Based on the present discovery and using techniques described herein and known in the art, analysis of PFRs and protein units in various combinations for similar different, and synergistic correlations to drug effects and diseases can identify PFRs, protein units and sets of protein units that have identified significance in combination.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease, condition, or disorder. The term “patient” includes human and veterinary subjects. The disclosed methods are particularly useful for human subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist.”

The term “inhibit” means to reduce or decrease in activity or expression. This can be a complete inhibition or activity or expression, or a partial inhibition Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity or effect can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The disclosed methods include the determination, identification, indication, correlation, diagnosis, prognosis, etc. (which can be referred to collectively as “identifications”) of subjects, diseases, compounds, effects, conditions, states, etc. based on measurements, detections, comparisons, analyses, assays, screenings, etc. For example, identifying subjects, specific drug effect-correlated protein sub-regions, and identifying drugs correlated with specific protein sub-regions, all based on the discovered correlation of drug effects with genetic alterations in specific sub-regions of proteins, are useful improving treatment of disease. Other examples include identifying a compound as a protein unit-specific compound, identifying a drug-specific set of protein units for a compound and a disease, identifying a correlation between genetic features in the test set of protein units and the effect of the compound on a disease, identifying the test set of protein units as a drug-specific set of protein units for the compound and for the disease, identifying the compound as a protein unit/disease-associated compound for the disease and for the test set of protein units, identifying protein unit-specific compounds for a set of protein units and a disease, identifying a correlation between genetic features in the set of protein units and the effect of a test compound on a disease, identifying the PFR of the protein as a drug-specific PFR for the compound and for the disease, and identifying the compound as a PFR/disease-associated compound for the disease and for the PFR of the protein.

Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing or that could benefit from the treatment will receive it and subjects that do not need or would not benefit from the treatment will not receive it.

Accordingly, also disclosed herein are methods comprising taking particular actions following and based on the disclosed identifications. For example, disclosed are methods comprising creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods comprising treating, monitoring, following-up with, advising, etc. a subject identified in any of the disclosed methods. Also disclosed are methods comprising treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component or characteristic (and/or a subject for which a record has been made of such an identification) can be treated with a therapy based on or directed to the high level component or characteristic. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compounds of the invention.

By the term “effective amount” of a compound as provided herein is meant a nontoxic but sufficient amount of the compound to provide the desired result. The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need of treatment for a disease or condition. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

Any of the identified compounds can be used therapeutically in combination with a pharmaceutically acceptable carrier. The compounds can be conveniently formulated into pharmaceutical compositions composed of one or more of the compounds in association with a pharmaceutically acceptable carrier. See, e.g., Remington's Pharmaceutical Sciences, latest edition, by E. W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a correlation assessment is disclosed and discussed and a number of modifications that can be made to the steps and components are discussed, each and every combination and permutation of the steps and components and of the modifications that are possible are specifically contemplated unless specifically indicated to the contrary. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to “the cell” is a reference to one or more cells and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different protein units does not indicate that the listed protein units are obvious one to the other, nor is it an admission of equivalence or obviousness.

### EXAMPLES

### Example 1

### Analysis of Individual Protein Regions Provides New Insights on Cancer Pharmacogenomics

There is a need for better translation of genomic and pharmacologic data on cancer and other diseases into meaningful and clinically relevant hypothesis is data analysis. While numerous methods have been applied to the analysis of such datasets, most of them, particularly those dealing with mutation data, use a protein-centric perspective, as they do not take into account the specific position of the different mutations within a protein. Such approaches have been proven useful in many applications; however, they cannot fully deal with situations in which different mutations in the same protein have different effects depending on which region of the protein is being altered.

The present study demonstrates that such protein-centric analyses of genetic alterations miss subtler, yet still relevant, effects mediated by mutations in specific protein regions. Using datasets on the genomics of cancer cell lines and the effect of drugs on the cancer cell lines, analysis of genetic alterations in specific protein regions and correlation of such region-level genetic alterations with drug effects was performed. The results show that protein region-level genetic alterations are correlated with drug effects, including many cases where the genetic alterations averaged over the protein as a whole did not show correlation with drug effects. This provides richer and more effective information on drugs and their effects on cancer.

### 1. Materials and Methods

i. Cell Line Mutations

The CCLE (Cancer Cell Line Encyclopedia; website broadinstitute.org/ccle; Barretina et al., Nature 483:603-607 (2012)) dataset, which includes the mutation profiles of 1,668 genes in 906 human cancer cell lines and drug activity data for 24 different anticancer compounds, was used in the present study. The analysis was focused on missense mutations, as truncating mutations can sometimes be misleading when performing the analysis in terms of functional regions. For example, when analyzing a protein that contains two different domains, if a truncating mutation happens in the first domain, it is not obvious whether the functional consequences of the mutation are caused by the fact that the first domain is altered or that the second domain is missing. The missense mutations reported by CCLE were mapped from their genomic coordinates to every protein coding isoform from ENSEMBL using the Variant Effect Predictor tool (McLaren et al., Bioinformatics 26:2069-2070 (2010)). From the original 42,603 genomic-point mutations in 1,668 genes, 156,817 protein missense mutations were obtained in 9,311 proteins.

ii. Drug Activity Data

CCLE contains data on the drug activity of 24 different compounds in 479 cell lines from 8-point dose-response curves. These curves are adjusted to a logistical-sigmoidal function and described by 4 different variables: the maximal effect level (Amax), the drug concentration at half-maximal activity of the compound (EC50), the concentration at which the drug response reached an absolute inhibition of 50% (IC50), and the activity area, which is the area above the dose-response curve. In our analysis only the activity area was used because, according to the CCLE, it captures simultaneously both variables of drug activity: its efficacy and its potency.

iii. Protein Functional Regions

For the present study, protein functional regions were defined as domains or intrinsically disordered regions. Intrinsically disordered regions were included because these can also contain important functional regions such as phosphorylation sites or regions that regulate or mediate protein interactions (Dunker et al., FEBS J 272:5129-5148 (2005)). To identify protein domains, annotated Pfam domains were retrieved from ENSEMBL for each protein isoform. A set of 1,300 potential domains identified by AIDA (ab initio domain assembly; Xu et al., Nucleic Acids Research 12:W308-W313 (2014) (Web Server issue); Internet site ffas.burnham.org/AIDA/), an algorithm based on remote homology, were also included. Foldindex (Prilusky et al., Bioinformatics 21(16):3435-8 (2005)) was used to predict intrinsically disordered regions for each protein. Those regions with a predicted unfolded score below −0.1 were included in the present study.

The different mutations of each cell line were mapped to these protein features, giving a total of 30,798 altered regions in 906 cell lines. These regions are divided into 19,918 Pfam domains and 10,880 intrinsically disordered regions. Note that the features can overlap, as the predictions were performed independently and there is no reason why, for example, an intrinsically unfolded region cannot overlap with (or even be located within) a Pfam domain. Note also that these numbers refer to PFRs in all known protein isoforms according to ENSEMBL v72. While the results for all these PFR-Drug pairs can be browsed at the website cancer3d.org, in this example only discuss results obtained for the largest isoform of each protein.

iv. Identification of PFR Perturbations Correlating with Drug Activity

The e-Drug analysis protocol looks for PFRs that, when mutated, correlate with drug activity of the different drugs. The cell lines were divided into those that have a coding missense mutation in the region being studied and those that do not. A Wilcoxon test was then performed comparing the drug activity of each compound in the two groups and kept those with a p-value below 0.01. Finally, for those gene regions associated to a certain drug, the activity of the cell lines mutated in the region of interest was compared to the activity of cell lines mutated in other regions of the gene. By doing this, regions that are significantly different from the rest of the gene were identified. In this case, since the number of cell lines in both groups is lower and fewer tests were performed, a significance threshold of 0.05 instead of 0.01 was established. True positives were considered those PFR that passed both thresholds and that are not in proteins that show an association (p<0.01) with the same drug at the whole-protein level. Note that the analysis is performed independently for each PFR. In the case that a protein contains two overlapping regions, the e-Drug analysis protocol will handle each one of them independently and return their corresponding results.

v. Statistical Significance Analysis

One of the problems that arise when analyzing PFRs instead of whole proteins is that the statistical power of the sample decreases significantly, as (I) there are less cell lines with mutations in the individual regions and (II) the number of correlations being tested increases, making multiple-testing corrections more stringent. To overcome these limitations and decrease the number of false positives among the associations three different thresholds were required for an association to be considered positive (see

FIG. 1). First, the p value of comparing the activity of the drugs between cell lines with mutations in the PFR against those without them has to be below 0.01. This left 350 potential PFR-drug pairs identified in the CCLE data. Then, the analysis was repeated at the protein level and all the pairs that were also identified there were removed (p<0.01, n=102, Figure lf). Finally, for the remaining 248 pairs, the drug activity of the cell lines was compared with mutations in the PFR against cell lines with mutations in other regions of the same protein.

vi. Protein Expression Data from TCPA

Expression data for 461 different proteins in 93 cancer cell lines was downloaded from the TCPA (The Cancer Proteome Atlas; Internet site appl.bioinformatics.mdanderson.org/tcpa/_design/basic/index.html; Cancer Genome Atlas Research et al., Nat Genet 45:1113-1120 (2013)) website on Dec. 11, 2013. Cell line names used in TCPA were manually mapped to CCLE when automated mapping was not possible.

In order to find proteins with altered expression or phosphorylation levels in cell lines with mutations in PFRs of interest cell lines, the proteins were grouped according to the mutation status of such PFRs and compared the expression levels in each group using a Wilcoxon test. To find proteins whose expression correlated with the activity of anticancer drugs a Pearson correlation test using R was performed.

vii. TCGA Survival Analysis

Both clinical and mutation data for the 3,205 tumors described in the pan-cancer analysis of the TCGA were downloaded. Data from patients that had not been treated with any of the drugs included in the CCLE was then filtered out. Since most drugs included in the CCLE are still in under clinical research, there were only enough patients to analyze 2 different drugs: paclitaxel (n=778) and irinotecan (n=58). Each of these subsets of patients have then been classified in 3 groups: those that have a mutation in a PFR that, according to the analysis, increases resistance to the drug used to treat them; those with mutations in other regions of the same genes; and those with no mutations in these genes.

The analysis was limited to gene-regions associated with lower drug activity because there are more such regions as compared to regions associated with increased activity. As a result very few patients in the TCGA dataset carry mutations in the former type of regions and were treated with the matching drug. The survival analysis was performed using the “Survival” package for R.

viii. Protein-Drug Interaction Data

It would be natural to expect that proteins that are associated with drug phenotypes might be enriched in either drug targets or their partners. To determine this, the STITCH database that contains information on protein—chemical interactions was downloaded. The known protein interactions for each drug were retrieved and the overlap of proteins on this list was compared with the proteins that showed an association with that same drug according to analysis with the Fisher test. The analysis was performed using three different thresholds for the protein-drug interaction score as reported in STITCH: 700, 800 and 900. The analysis was also extended to (a) proteins interacting with drug targets (according to human protein reference database (HPRD; Peri et al., Genome Research 13:2363-71 (2003); website hprd.org/), BioGRID (Stark et al., Nucleic Acids Research 1(34): 535-539 (2006); Internet site thebiogrid.org), Molecular INTeraction database (MINT; Chatr-aryamontri et al., Nucleic Acids Res. 35:D572-D574 (2007) (Database issue); Internet site mint bio.uniroma2.it/mint/Welcome.do), or Database of Interacting Proteins (DiP; Xenarios et al., Nucleic Acids Research 30(1):303-5 (2002); Internet site dip.doe-mbi.ucla.edu/dip/Main.cgi)) and to (b) proteins that bind chemicals with a similar structure. These druglike chemicals were defined as those that have a Tanimoto 2D similarity score with the drug over 0.70. The Tanimoto scores were calculated with the R package RCDK.

### 2. Results

i. Analysis Schema and Overall Results

The e-Drug analysis protocol introduced here is illustrated in FIG. 1 for the ERBB3 protein and the c-Met inhibitor PF2341066. Some of the many functional relationships of this protein include physical interactions (with EGFR, NRG1 and JAK3) or phosphorylations (by CDKS or ERBB3 itself). All these relationships can be mapped to specific PFRs within ERBB3. For example, the N-terminal EGF receptor domains mediate the interactions with EGFR and NRG1 (shown in medium dark gray (panel b) in FIG. 1), whereas ERBB3′s kinase domain interacts with JAK3 and phosphorylates other ERBB3 molecules (shown in dark gray (panel b) in FIG. 1).

When using the protein level analysis, cell lines with mutations in ERBB3 do not show any bias in the activity of PF2341066, suggesting that mutations in this protein do not influence the sensitivity towards this drug. However, the PFR level analysis shows that cell lines with mutations in the receptor domain are resistant to treatment with inhibitor, while those with mutations in any other PFR of this protein, such as the kinase domain, do not show any specific behavior.

Following the e-Drug analysis protocol, 171 statistically significant PFR-drug associations were identified (p<0.05 in the comprehensive, multistage significance analysis as described in the Methods Section). The full list is provided in the Table 2 and is available on-line from a newly developed resource at the website cancer3d.org.

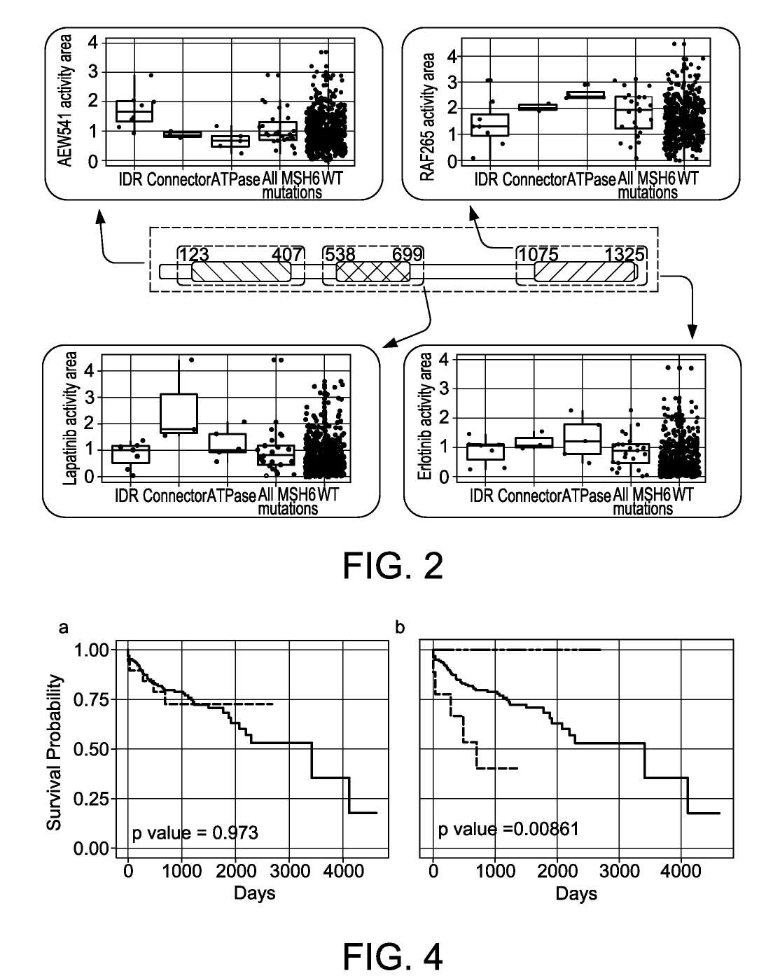

Some cases were found where PFR perturbations associated with different drugs belong to the same protein. For example, the MSH6 protein contains 3 different PFRs associated with 3 different drugs (FIG. 2). Mutations in the N-terminal intrinsically disordered region (IDR) of this protein are associated with increased AEW541 activity, while mutations in the connector (Pf05188) and ATPase (Pf00488) domains are associated with higher Lapatinib and RAF265 activities respectively. Interestingly, there are some references in the literature that are consistent with the discovered interaction between RAF265 and MSH6. Given that MSH6 has been recently shown to be involved in pathways related to the repair of DNA-double-strand breaks (Shahi et al., Nucleic Acids Res 39:2130-2143 (2011)), the association identified here between mutations in MSH6′s ATPase domain, as well as other PFRs in PAXIP1 or TP53, and the activity of RAF265 indicate that the DNA-damage response pathway can have a role in modulating the activity of this drug.

ii. Integration of CCLE with other Molecular Datasets Provides Further Insights into the Role of Individual PFRs

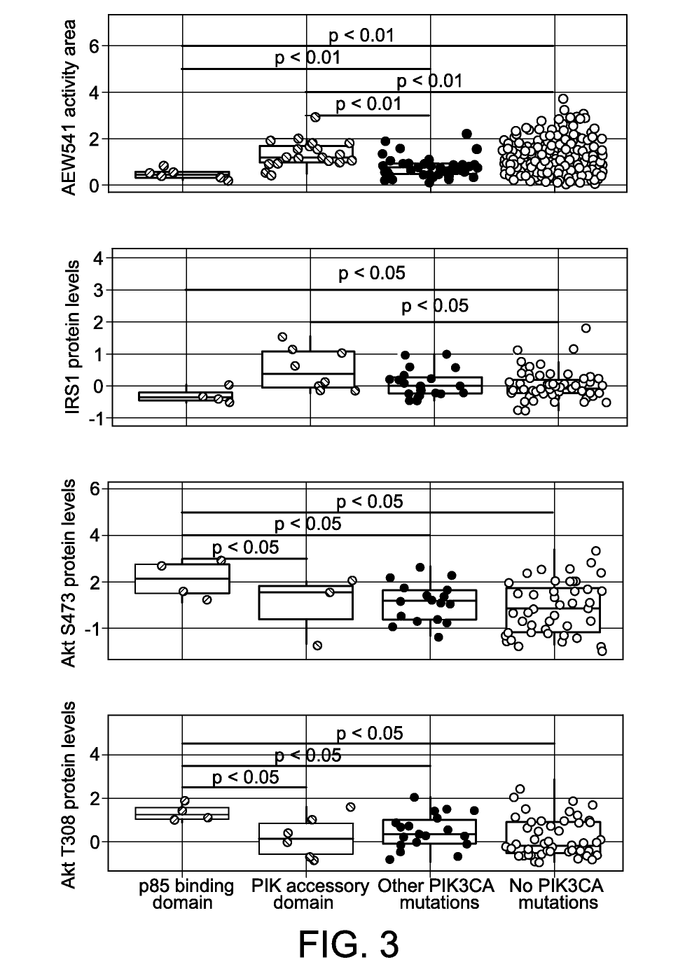

The best examples of the advantages of studying mutation effects on individual PFRs are those where mutations in different regions of the same protein are associated with the same drug but in opposite directions. This is the case for PIK3CA and the IGF1R inhibitor AEW541. Using the e-Drug analysis protocol mutations in the p85 binding domain (Pf02192) were found to decrease the activity of the AEW541 while mutations in the PIK accessory domain (Pf00613) were found to be associated with increased activity of the same drug (FIG. 3). Mutations in different regions of PIK3CA are known to be oncogenic through different molecular mechanisms (Burke et al., Proc Natl Acad Sci USA 109:8 (2012)), which is consistent with the opposite effects in AEW541 sensitivity observed for these two domains.

To find features that could explain the different responses to AEW541 depending on the PIK3CA domain mutated, proteomics data from The Cancer Proteome Atlas (Li et al., Nat Methods 10:1046-1047 (2013)) were used. The analysis was focused on IRS1 expression levels as well as Akt phosphorylation status in the cell lines with mutations in the two PIK3CA domains, because these proteins are immediately up and downstream from PIK3CA respectively.

Cell lines with mutations in the PIK accessory domain did not have changes in the phosphorylation levels of Akt at either T308 (p>0.34) or 5473 (p>0.07), but did have higher IRS 1 expression (p<0.05) (FIG. 3). These results are consistent with recent data showing that the E545K mutation in PIK3CA enhances its interaction with IRS1 (Hao et al., Cancer Cell 23:583-593 (2013)). Since IRS1 mediates the interaction between IGF1R and PIK3CA, this increased interaction with IRS1 (and therefore dependence on interaction with receptor tyrosine kinases such as IGF1R) can explain why cell lines with mutations in Pf00613 are more sensitive to IGF1R inhibition.

On the other hand, cell lines with mutations in the p85 binding domain showed higher Akt phosphorylation levels at both, T308 (p<0.01) and S473 (p<0.02), and also had lower IRS1 protein levels (p<0.01) (FIG. 3). Since Akt is one of the main PIK3CA effectors, these results could mean that cell lines with mutations in the p85-binding domain have intrinsically active PIK3CA phosphorylation activity, regardless of its interaction with receptor tyrosine kinases such as IGF1R. In this scenario, inhibiting IGF1R with AEW541 would have little effect, as these cells are already signaling downstream towards Akt.

Putting these results together, mechanisms for the two PFR-AEW541 associations can be proposed. First, AEW541 inhibits the kinase domain of IGF1R. In those cell lines with mutations in the PIK domain of PIK3CA, there is a gain of interaction between this protein and IRS1. This will likely increase the signaling through IGF1R, explaining why cell lines with mutations in this domain are more sensitive to the inhibition of this receptor. On the other hand, cell lines with mutations in the p85-binding domain have lower IRS1 expression and higher AKT1 phosphorylation levels. Together, this indicates that PIK3CA is active independently of its interaction with extracellular receptors, signaling directly downstream towards AKT1. This would explain why these cells are resistant to AEW541.

Given recent concerns about pharmacogenomic data using cell lines (Haibe-Kains et al., Nature 504:389-393 (2013)), these results were reproduced in human tumors also analyzed by TCPA (n=2229). All the protein changes caused by PIK3CA mutations were confirmed, as tumors with mutations in Pf02192 have higher levels of Akt phosphorylation at both T308 and 5473. These samples also have lower IRS1 levels than those with Pf00613 or no mutations at all. Tumor samples with mutations in Pf00613, on the other hand, have higher IRS1 levels and no changes in Akt phosphorylation status.

iii. Drug-PFR Correlations Predict Success of Cancer Treatment

After confirming in tumor samples the molecular mechanisms underlying the PFR-drug associations between AEW541 and PIK3CA, the PFRs identified in the CCLE data were used to predict survival of actual cancer patients. To that end, clinical data from patients whose tumors have been analyzed by The Cancer Genome Atlas (TCGA) (Cancer Genome Atlas Research et al., Nat Genet 45:1113-1120 (2013)) were used to find patients that had been treated with drugs included in the CCLE. Since most of these drugs are still under clinical research, there were sufficient data only to analyze two types of drugs: Paclitaxel (n=778) and the Topoisomerse inhibitors Irinotecan and Topotecan (n=188). Genomic data of the patients was used to find those who had mutations in PFRs that are associated with increased resistance towards these drugs (FIG. 4). While no differences were found in patients treated with paclitaxel (p>0.9), patients that had mutations in PFRs associated with resistance to Topoisomerase inhibitors had worse outcomes (p<0.01) than those with mutations in other regions of the same proteins or no mutations in these proteins at all. Interestingly, the mutation status of the whole proteins that contain the associated PFRs cannot predict the outcome of the patients (p>0.9), indicating that only mutations in the specific PFRs, but not in other regions of the same proteins, confer resistance to Topoisomerase inhibitors.

iv. Proteins and PFRs Associated with Drugs do not Usually Overlap with Drug Targets

One of the possible mechanisms for a PFR to be associated with differential drug activity is that the protein itself directly interacts with the drug of interest. To explore this hypothesis, the set of proteins associated with each drug was compared, at both whole-protein and individual PFR levels, to the set of drug targets as identified by the STITCH database (Kuhn et al., Nucleic Acids Res 40:D876-880 (2012)). Of the 19 drugs that had at least one known target, only AZD6244 had its associated proteins and PFRs enriched with its targets, as mutations in two of the five genes known to code for proteins interacting directly with the drug, BRAF and KRAS, are also associated with differential activity for this drug (p<0.005). Expanding the search by varying the STITCH interaction score, including proteins that interact with compounds that have similar structures to the drugs included in the analysis (Tanimoto score>0.70) or to proteins interacting with the drug targets, also did not show any statistically significant associations.

v. Gene Set Enrichment Analysis of the PFRs and Proteins Correlating with Drug Activity Reveals Common Functions

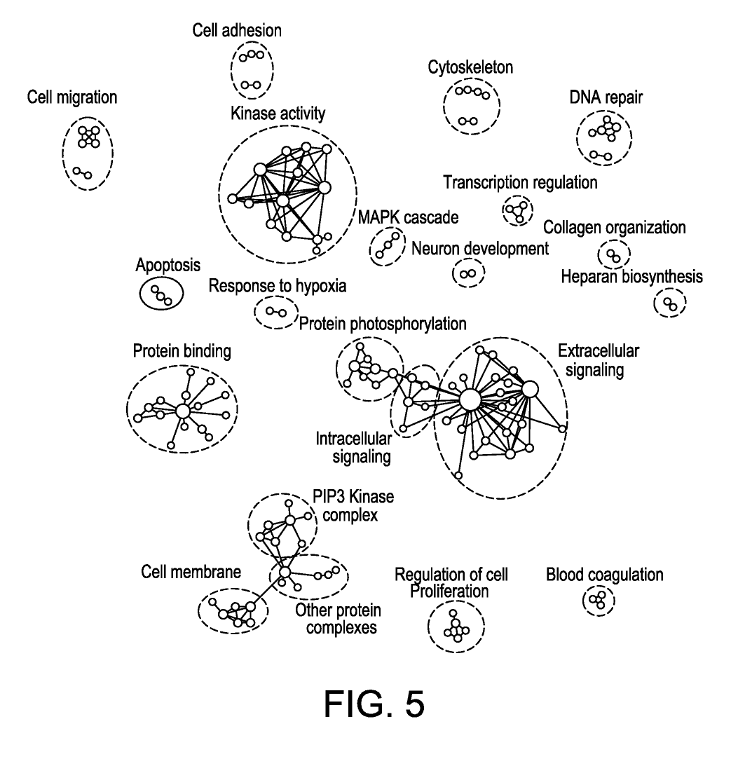

A gene set enrichment analysis was performed using Gene Ontology (GO) annotations downloaded from Uniprot (website uniprot.org/help/gene_ontology) to understand the shared functions and relationships of the proteins and regions associated with changes in drug activity (FIG. 5). Several groups of GO terms identified in this analysis, such as those related to signaling cascades (extracellular and intracellular signaling), signal transduction (kinase activity or protein phosphorylation), or protein binding, indicate that these genes can be involved in either the same pathways targeted by the drugs or similar pathways that might have some level of redundancy. Other GO terms, such as apoptosis, regulation of cell proliferation, or response to hypoxia, are functions known to play a role in drug resistance and carcinogenic potential of cells.

Another group of GO terms identified in the analysis are those associated with the cytoskeleton. Given that most of the drugs analyzed in this study (17 out of 24) are kinase inhibitors, this was an unexpected observation. However, there is some evidence of the relationship between cytoskeleton proteins and the activity of kinase inhibitors in the literature. For example, many receptor tyrosine kinases, such as EGFR, HER2, IGF 1R, or FGFR, undergo internalization upon ligand binding. Moreover, interactions between Erlotinib and MYO2 or MYH9 have been described, and a MYH9 inhibitor synergizes with EGFR inhibitors to induce apoptosis in cells carrying the drug-resistant mutation T790M (Chiu et al., Mol Oncol 6:299-310 (2012)).

### 3. Discussion

Identifying biological features that correlate with the activity of anticancer drugs has been the subject of a significant and growing research focus in recent years. However, most of these efforts do not take into account the modular nature of proteins and focus on perturbations at the whole-protein level. Such analyses are doomed to miss cases in which the location of the mutation within the protein influences its effects. The present study is the first systematic analysis of drug activity associations that distinguishes between different functional regions within proteins. By focusing on specific PFRs, 171 associations have been shown between mutations in specific protein regions and changes in the activity of anticancer drugs. These associations could not have been identified by protein-centric approaches, as cell lines carrying mutations in other PFRs of the same protein (i.e. perturbing regions that mediate other functions) are not associated with any drug phenotype, thus adding noise to the analysis and making it impossible to identify the association.

Some cases were found in which the same gene is associated with different drugs through different PFRs, as in the case of MSH6 and the kinase inhibitors Erlotinib, AEW541, Lapatinib, and RAF265. The identification of such associations can provide insights into the putative mechanisms of the drug pleiotropy of a given gene, aiding in defining further experiments. A variation of this category is the association between PIK3CA and the AEW541, where mutations in different PFRs can have opposing effects in terms of the activity of the drug.

The practical value of the PFR-drug associations discovered here on the independent data from the TCGA consortium was also shown. Specifically, it was shown that patients from the TCGA harboring mutations in regions associated with resistance to the drugs used to treat them have lower survival rates than patients with mutations in the very same genes but in regions not showing any association to the activity of such drugs. This result not only provides evidence of the significance of the e-Drug approach, but it also argues in favor of the value of drug activity data collected using cell lines (such as cell lines in the CCLE), an issue that has recently drawn significant attention (Haibe-Kains et al., Nature 504:389-393 (2013)). Another interesting result is that the proteins coded by genes associated with different drugs, regardless of the level of the analysis, do not seem to bind directly to the drugs themselves nor interact directly with the drug targets. This observation indicates that these genes modify drug activity through indirect interactions. For example, mutations in genes related to the cytoskeleton (a subset enriched in the genes identified in our analysis) might alter the potency of kinase inhibitors by changing the trafficking pattern of receptor tyrosine kinases. Such identifications are useful result of the eDrug analysis protocol.

Overall, this work expands the number of correlations between cancer somatic mutations and drug activity, thus increasing the information that can be extracted from every dataset. Focusing on PFRs, corresponding to protein domains or IDRs, provides better statistical results than analysis of individual mutations and allows identification of correlations in cases where different effects cancel out and thus are missed on the whole gene analysis level. At the same time, it provides more details about the mechanism of the drug resistance than the analysis on the gene level. Increasing the number and details of features that predict the activity of anticancer drugs has important consequences in the field of personalized medicine, as it increases accuracy in stratifying patients into groups that require different treatment regiments and can suggest drug combinations as exemplified for EGFR and MYH9.

One interesting direction of work refers to the interaction between multiple drug activity modifiers. Given the discovery of alterations that alter a cell's sensitivity towards a drug using the PFR-centric approach, correlations of multiple such alterations in the same cell line or patient can be identified. As described herein, sets of protein units (PFRs, PFR groups, and whole proteins) can be identified as drug- and disease-associated and used for making treatment decisions. Analysis of the relationship of different PFRs or different protein units can identify PFRs and protein units that have opposite effects (e.g., opposite correlations). Different PFRs and protein units can have similar, different, or synergistic relationships to drug effects and diseases. Most attempts to answer these challenging questions in the past were based on machine learning approaches (Costello et al., Nat Biotechnol doi:10.1038/nbt.2877 (2014)) which, given the multidimensional nature of the problem, seems to be the most natural approach. However, simple methods based on naively counting the presence or absence of specific alterations, such as the analysis of TCGA clinical data for Irinotecan and Topotecan presented here or analyses based on synthetic lethal interaction networks (Jerby-Amon et al., Cell 158:1199-1209 (2014)), have some predicting power. Regardless of the specific approach, these correlations can be used to advance the promise of personalized medicine.

Another generalization that comes from the discoveries described here is that data obtained using gene knockouts, silencing RNAs, or other technologies that completely abolish the activity of individual proteins will often miss more subtle effects caused by modifications of specific PFRs and other protein units. Finally, it bears emphasis that, just like the analyses at the protein level is not limited to the identification of features that correlate with drug activity, the analysis of PFR and protein unit perturbations can be useful when looking for features associated with any phenotype.

Consistent with the benefits of the eDrug analysis protocol and the PFR/drug correlations identified using the disclosed methods, identification of drug-specific PFRs and of PFR-specific drugs provides benefits, uses, and utilities beyond either identification of a specific genetic feature correlated with a drug or identification of the gene containing the specific genetic feature as relevant to the drug.

TABLE 2

| | | | | | | | pRest | pWhole | |
|---|---|---|---|---|---|---|---|---|---|
| Symbol | PFR | Start | End | Drug | Effect | pWT | Protein | Protein | ENSP |
| MAP3K1 | PF00069 | 1245 | 1508 | Lapatinib | 2.307 | 0.002 | 0 | 0.79 | ENSP00000382423 |
| MAP3K1 | PF07714 | 1246 | 1503 | Lapatinib | 2.307 | 0.002 | 0 | 0.79 | ENSP00000382423 |
| MSH6 | IDR | 123 | 407 | AEW541 | 1.592 | 0.005 | 0 | 0.717 | ENSP00000234420 |
| CACNB2 | PF00625 | 280 | 460 | L-685458 | 2.149 | 0.008 | 0.001 | 0.816 | ENSP00000320025 |
| ADAM22 | IDR | 148 | 248 | TKI258 | 0.303 | 0.005 | 0.001 | 0.109 | ENSP00000265727 |
| TPR | IDR | 1818 | 2102 | ZD-6474 | 1.675 | 0.001 | 0.001 | 0.386 | ENSP00000356448 |
| AFF4 | IDR | 334 | 699 | PD-0325901 | 2.491 | 0.003 | 0.001 | 0.163 | ENSP00000265343 |
| HDAC4 | IDR | 76 | 288 | Sorafenib | 1.809 | 0.01 | 0.001 | 0.725 | ENSP00000264606 |
| PRKG1 | PF00027 | 137 | 218 | Sorafenib | 0.177 | 0.006 | 0.001 | 0.763 | ENSP00000363092 |
| DAPK1 | PF01163 | 38 | 151 | PHA-665752 | 0.165 | 0.004 | 0.002 | 0.617 | ENSP00000418885 |
| ITGB4 | PF00041 | 1221 | 1309 | TAE684 | 0.229 | 0.004 | 0.002 | 0.903 | ENSP00000200181 |
| LAMA1 | PF00054 | 2514 | 2657 | AEW541 | 2.164 | 0.003 | 0.002 | 0.645 | ENSP00000374309 |
| LAMA1 | PF02210 | 2514 | 2653 | AEW541 | 2.164 | 0.003 | 0.002 | 0.645 | ENSP00000374309 |
| TTN | PF00041 | 28254 | 28339 | Topotecan | 1.485 | 0.002 | 0.002 | 0.157 | ENSP00000467141 |
| MTOR | IDR | 1442 | 1492 | Topotecan | 1.779 | 0.007 | 0.002 | 0.901 | ENSP00000354558 |
| PIK3CA | PF00613 | 520 | 703 | AEW541 | 1.301 | 0.01 | 0.002 | 0.729 | ENSP00000263967 |
| DAPK1 | IDR | 252 | 322 | PLX4720 | 4.893 | 0.001 | 0.002 | 0.817 | ENSP00000418885 |
| SETDB1 | PF00856 | 814 | 1266 | PF2341066 | 0.232 | 0.002 | 0.003 | 0.162 | ENSP00000271640 |
| SETDB1 | PF00856 | 814 | 1266 | TAE684 | 0.315 | 0.003 | 0.003 | 0.217 | ENSP00000271640 |
| LAMA1 | PF00054 | 2514 | 2657 | PF2341066 | 2.119 | 0.002 | 0.003 | 0.135 | ENSP00000374309 |
| LAMA1 | PF02210 | 2514 | 2653 | PF2341066 | 2.119 | 0.002 | 0.003 | 0.135 | ENSP00000374309 |
| DPYD | PF01207 | 644 | 733 | TKI258 | 0.348 | 0.003 | 0.003 | 0.594 | ENSP00000359211 |
| MAP3K13 | PF07714 | 172 | 406 | RAF265 | 0.375 | 0.008 | 0.003 | 0.281 | ENSP00000265026 |
| MAP3K13 | PF00069 | 171 | 406 | RAF265 | 0.375 | 0.008 | 0.003 | 0.281 | ENSP00000265026 |
| TNK2 | PF00069 | 190 | 442 | TKI258 | 0.356 | 0.01 | 0.003 | 0.846 | ENSP00000371341 |
| LRP1B | Q9NZR2.4468.4599 | 4468 | 4599 | Sorafenib | 0.179 | 0.002 | 0.003 | 0.43 | ENSP00000374135 |
| CDH2 | PF01049 | 748 | 903 | 17-AAG | 1.591 | 0.004 | 0.003 | 0.274 | ENSP00000269141 |
| PI4KA | PF00454 | 1846 | 2050 | PD-0325901 | 0.106 | 0.01 | 0.003 | 0.051 | ENSP00000255882 |
| TPR | IDR | 1818 | 2102 | TKI258 | 1.659 | 0.003 | 0.003 | 0.177 | ENSP00000356448 |
| TTN | PF00041 | 33395 | 33479 | PHA-665752 | 0.349 | 0.006 | 0.003 | 0.037 | ENSP00000467141 |
| INSRR | PF07714 | 980 | 1244 | PD-0332991 | 0.226 | 0.004 | 0.003 | 0.311 | ENSP00000357178 |
| INSRR | PF00069 | 980 | 1244 | PD-0332991 | 0.226 | 0.004 | 0.003 | 0.311 | ENSP00000357178 |
| TTN | PF00041 | 28254 | 28339 | Lapatinib | 1.883 | 0.003 | 0.003 | 0.118 | ENSP00000467141 |
| EPHA5 | PF01404 | 60 | 233 | Nutlin-3 | 0.16 | 0.004 | 0.004 | 0.108 | ENSP00000273854 |
| AFF4 | IDR | 334 | 699 | AZD6244 | 2.839 | 0.002 | 0.004 | 0.804 | ENSP00000265343 |
| MYC | IDR | 1 | 68 | AZD0530 | 0.094 | 0.002 | 0.004 | 0.362 | ENSP00000367207 |
| CREBBP | PF08214 | 1345 | 1639 | AZD6244 | 0.374 | 0.009 | 0.004 | 0.205 | ENSP00000262367 |
| PAPPA | Q13219.667.923 | 667 | 923 | LBW242 | 0.232 | 0.005 | 0.004 | 0.904 | ENSP00000330658 |
| TTN | PF00041 | 28254 | 28339 | Nilotinib | 2.069 | 0.004 | 0.004 | 0.602 | ENSP00000467141 |
| CLTCL1 | PF00637 | 979 | 1119 | TAE684 | 2.205 | 0.009 | 0.005 | 0.618 | ENSP00000445677 |
| PIK3CA | PF02192 | 32 | 108 | AEW541 | 0.441 | 0.005 | 0.005 | 0.729 | ENSP00000263967 |
| GUCY2C | PF00211 | 816 | 1002 | PHA-665752 | 0.19 | 0.006 | 0.005 | 0.184 | ENSP00000261170 |
| HDAC4 | IDR | 76 | 288 | TKI258 | 1.926 | 0.008 | 0.006 | 0.887 | ENSP00000264606 |
| MECOM | IDR | 897 | 1184 | ZD-6474 | 0.314 | 0.007 | 0.006 | 0.091 | ENSP00000417899 |
| BCR | PF00620 | 1068 | 1217 | TAE684 | 0.127 | 0.006 | 0.006 | 0.264 | ENSP00000303507 |
| SMG1 | IDR | 1 | 172 | LBW242 | 0.122 | 0.007 | 0.006 | 0.23 | ENSP00000374118 |
| TIAM1 | PF00621 | 1044 | 1233 | L-685458 | 2.788 | 0.005 | 0.006 | 0.139 | ENSP00000286827 |
| TTN | PF00041 | 30721 | 30807 | RAF265 | 2.254 | 0.006 | 0.007 | 0.135 | ENSP00000467141 |
| TTN | PF07679 | 4993 | 5069 | PF2341066 | 0.131 | 0.007 | 0.007 | 0.684 | ENSP00000467141 |
| TTN | PF07686 | 4990 | 5059 | PF2341066 | 0.131 | 0.007 | 0.007 | 0.684 | ENSP00000467141 |
| TP53 | PF07710 | 318 | 358 | RAF265 | 0.485 | 0.006 | 0.007 | 0.023 | ENSP00000269305 |
| BIRC6 | Q9NR09.1083.1222 | 1083 | 1222 | Nutlin-3 | 2.492 | 0.009 | 0.007 | 0.907 | ENSP00000393596 |
| TPR | IDR | 1818 | 2102 | Lapatinib | 1.909 | 0.006 | 0.007 | 0.03 | ENSP00000356448 |
| ADAM22 | IDR | 148 | 248 | Nilotinib | 0.137 | 0.009 | 0.007 | 0.271 | ENSP00000265727 |
| PPARGC1A | IDR | 279 | 373 | Panobinostat | 0.731 | 0.008 | 0.007 | 0.298 | ENSP00000264867 |
| TG | P01266.1695.1822 | 1695 | 1822 | Panobinostat | 0.724 | 0.005 | 0.007 | 0.248 | ENSP00000220616 |
| MYC | IDR | 1 | 68 | TAE684 | 0.169 | 0.008 | 0.007 | 0.602 | ENSP00000367207 |
| CSMD3 | PF00084 | 2694 | 2748 | PD-0325901 | 0.253 | 0.007 | 0.007 | 0.696 | ENSP00000297405 |
| TTN | PF07679 | 35130 | 35218 | PHA-665752 | 0.075 | 0.009 | 0.008 | 0.037 | ENSP00000467141 |
| TTN | PF07679 | 32714 | 32792 | AZD0530 | 1.918 | 0.009 | 0.008 | 0.664 | ENSP00000467141 |
| NCOA2 | IDR | 1125 | 1280 | Erlotinib | 2.281 | 0.006 | 0.008 | 0.12 | ENSP00000399968 |
| PTK7 | PF07714 | 807 | 1069 | PD-0325901 | 2.082 | 0.006 | 0.008 | 0.617 | ENSP00000418754 |
| ALS2 | PF00621 | 695 | 878 | Panobinostat | 0.76 | 0.005 | 0.008 | 0.694 | ENSP00000264276 |
| CTTN | IDR | 114 | 294 | ZD-6474 | 0.267 | 0.005 | 0.008 | 0.153 | ENSP00000365745 |
| TNN | PF00041 | 622 | 697 | AEW541 | 0.261 | 0.008 | 0.008 | 0.515 | ENSP00000239462 |
| BAI3 | PF12003 | 586 | 808 | AZD0530 | 2.123 | 0.004 | 0.008 | 0.849 | ENSP00000359630 |
| ITGB1 | PF00362 | 34 | 464 | PF2341066 | 0.298 | 0.003 | 0.008 | 0.04 | ENSP00000364094 |
| EXT2 | PF03016 | 134 | 413 | TAE684 | 0.438 | 0.008 | 0.008 | 0.055 | ENSP00000379032 |
| TTN | PF07679 | 2971 | 3050 | Topotecan | 0.262 | 0.008 | 0.008 | 0.157 | ENSP00000467141 |
| TTN | PF00041 | 26686 | 26766 | 17-AAG | 1.499 | 0.008 | 0.009 | 0.523 | ENSP00000467141 |
| ADAM12 | PF01562 | 60 | 162 | Irinotecan | 0.423 | 0.008 | 0.009 | 0.903 | ENSP00000357668 |
| MYC | IDR | 1 | 68 | RAF265 | 0.231 | 0.002 | 0.009 | 0.038 | ENSP00000367207 |
| CPNE5 | Q9HCH3.492.561 | 492 | 561 | AZD0530 | 2.102 | 0.006 | 0.01 | 0.143 | ENSP00000244751 |
| TSSK1B | IDR | 274 | 367 | TAE684 | 0.3 | 0.008 | 0.01 | 0.203 | ENSP00000375081 |
| MSH5 | PF00488 | 561 | 794 | ZD-6474 | 0.266 | 0.005 | 0.01 | 0.351 | ENSP00000431693 |
| MSH5- | PF00488 | 561 | 794 | ZD-6474 | 0.266 | 0.005 | 0.01 | 0.351 | ENSP00000417871 |
| SAPCD1 | | | | | | | | | |
| TNNI3K | PF00023 | 303 | 334 | AEW541 | 0.234 | 0.007 | 0.01 | 0.128 | ENSP00000359928 |
| PCDH15 | PF00028 | 521 | 605 | Irinotecan | 0.433 | 0.008 | 0.01 | 0.249 | ENSP00000354950 |
| MLL3 | IDR | 2054 | 2236 | Lapatinib | 3.578 | 0.009 | 0.01 | 0.774 | ENSP00000347325 |
| LRP2 | PF00057 | 3718 | 3754 | PLX4720 | 3.241 | 0.009 | 0.01 | 0.746 | ENSP00000263816 |
| UBE3B | PF00632 | 737 | 1068 | Panobinostat | 1.246 | 0.005 | 0.01 | 0.551 | ENSP00000391529 |
| TTN | PF07679 | 7795 | 7885 | Topotecan | 0.435 | 0.009 | 0.01 | 0.157 | ENSP00000467141 |
| CACNB2 | PF00625 | 280 | 460 | AZD0530 | 2.746 | 0.004 | 0.01 | 0.138 | ENSP00000320025 |
| PRKG1 | PF00027 | 137 | 218 | TAE684 | 0.208 | 0.003 | 0.01 | 0.146 | ENSP00000363092 |
| NAV3 | Q8IVL0.1916.2020 | 1916 | 2020 | 17-AAG | 0.567 | 0.009 | 0.01 | 0.852 | ENSP00000381007 |
| MYH10 | PF00063 | 87 | 802 | TAE684 | 0.596 | 0.009 | 0.011 | 0.102 | ENSP00000353590 |
| NLRP3 | PF05729 | 220 | 389 | PD-0332991 | 0.229 | 0.008 | 0.011 | 0.109 | ENSP00000337383 |
| CNTRL | IDR | 1711 | 2049 | TAE684 | 0.216 | 0.004 | 0.011 | 0.202 | ENSP00000362962 |
| TAF1L | PF00439 | 1409 | 1488 | Panobinostat | 0.735 | 0.009 | 0.011 | 0.181 | ENSP00000418379 |
| PCDH15 | PF00028 | 824 | 916 | Nutlin-3 | 0.111 | 0.009 | 0.012 | 0.638 | ENSP00000354950 |
| CUBN | PF00431 | 817 | 925 | Nilotinib | 0.22 | 0.005 | 0.012 | 0.476 | ENSP00000367064 |
| PTPRT | PF00102 | 1224 | 1458 | Paclitaxel | 0.516 | 0.006 | 0.012 | 0.07 | ENSP00000362294 |
| FANCM | IDR | 1649 | 1795 | Nutlin-3 | 0.121 | 0.009 | 0.012 | 0.239 | ENSP00000267430 |
| RASA1 | PF00616 | 769 | 942 | PF2341066 | 0.103 | 0.006 | 0.012 | 0.802 | ENSP00000274376 |
| FPGT- | PF00023 | 303 | 334 | AEW541 | 0.234 | 0.007 | 0.012 | 0.036 | ENSP00000450895 |
| TNNI3K | | | | | | | | | |
| MYH10 | PF00063 | 87 | 802 | AZD0530 | 0.448 | 0.001 | 0.013 | 0.137 | ENSP00000353590 |
| GRIN2A | IDR | 947 | 1234 | AZD6244 | 1.863 | 0.007 | 0.014 | 0.052 | ENSP00000379818 |
| PLCG1 | IDR | 50 | 94 | PHA-665752 | 2.875 | 0.009 | 0.014 | 0.257 | ENSP00000244007 |
| PLCG1 | PF00169 | 40 | 140 | PHA-665752 | 2.875 | 0.009 | 0.014 | 0.257 | ENSP00000244007 |
| ZNF608 | IDR | 410 | 617 | Lapatinib | 2.164 | 0.008 | 0.015 | 0.093 | ENSP00000307746 |
| PTK7 | PF07714 | 807 | 1069 | AZD6244 | 2.189 | 0.008 | 0.016 | 0.373 | ENSP00000418754 |
| HIPK2 | PF00069 | 199 | 527 | TKI258 | 0.39 | 0.006 | 0.016 | 0.053 | ENSP00000385571 |
| TNK2 | PF00069 | 190 | 442 | Nutlin-3 | 0.106 | 0.002 | 0.016 | 0.074 | ENSP00000371341 |
| ADAMTS20 | PF01562 | 31 | 186 | AZD0530 | 0.229 | 0.004 | 0.016 | 0.073 | ENSP00000374071 |
| NRK | PF00780 | 1214 | 1549 | Irinotecan | 0.604 | 0.003 | 0.017 | 0.026 | ENSP00000438378 |
| AATK | IDR | 914 | 1030 | Lapatinib | 4.308 | 0.004 | 0.017 | 0.058 | ENSP00000324196 |
| PAXIP1 | IDR | 382 | 604 | RAF265 | 0.252 | 0.007 | 0.017 | 0.165 | ENSP00000380376 |
| MSH6 | PF05188 | 538 | 699 | Lapatinib | 3.569 | 0.009 | 0.017 | 0.061 | ENSP00000234420 |
| SMO | Q99835.555.638 | 555 | 638 | 17-AAG | 0.582 | 0.005 | 0.017 | 0.189 | ENSP00000249373 |
| GUCY2F | PF01094 | 75 | 408 | LBW242 | 3.016 | 0.001 | 0.017 | 0.13 | ENSP00000218006 |
| JAK1 | PF07714 | 876 | 1147 | ZD-6474 | 1.823 | 0.006 | 0.017 | 0.042 | ENSP00000343204 |
| JAK1 | PF00069 | 877 | 1147 | ZD-6474 | 1.823 | 0.006 | 0.017 | 0.042 | ENSP00000343204 |
| RASGRF2 | PF00621 | 249 | 426 | Paclitaxel | 0.57 | 0.008 | 0.018 | 0.119 | ENSP00000265080 |
| ROBO2 | PF00041 | 524 | 607 | PHA-665752 | 0.089 | 0.01 | 0.019 | 0.561 | ENSP00000327536 |
| ACOXL | PF01756 | 400 | 545 | AZD0530 | 1.962 | 0.009 | 0.019 | 0.464 | ENSP00000407761 |
| GTSE1 | Q9NYZ3.626.720 | 645 | 739 | PF2341066 | 2.245 | 0.008 | 0.019 | 0.08 | ENSP00000415430 |
| MYC | IDR | 1 | 68 | AZD6244 | 0.078 | 0.002 | 0.019 | 0.066 | ENSP00000367207 |
| TNK2 | PF00069 | 190 | 442 | ZD-6474 | 0.271 | 0.005 | 0.02 | 0.31 | ENSP00000371341 |
| ALK | Q9UM73.46.188 | 46 | 188 | Panobinostat | 0.783 | 0.008 | 0.02 | 0.37 | ENSP00000373700 |
| GUCY1A2 | PF00211 | 512 | 728 | LBW242 | 0.305 | 0.007 | 0.022 | 0.264 | ENSP00000282249 |
| NF1 | PF00616 | 1256 | 1451 | Panobinostat | 0.817 | 0.003 | 0.023 | 0.169 | ENSP00000351015 |
| COL3A1 | PF01410 | 1249 | 1465 | PHA-665752 | 0.247 | 0.008 | 0.023 | 0.103 | ENSP00000304408 |
| SRPK1 | IDR | 1 | 87 | Lapatinib | 4.489 | 0.003 | 0.024 | 0.158 | ENSP00000354674 |
| URB2 | Q14146.21.253 | 21 | 253 | RAF265 | 0.292 | 0.008 | 0.024 | 0.805 | ENSP00000258243 |
| PRKD3 | IDR | 320 | 391 | ZD-6474 | 0.197 | 0.008 | 0.024 | 0.184 | ENSP00000234179 |
| INSRR | PF01030 | 47 | 157 | Lapatinib | 0.285 | 0.007 | 0.024 | 0.197 | ENSP00000357178 |
| ALS2 | PF02204 | 1553 | 1653 | Lapatinib | 3.107 | 0.005 | 0.024 | 0.042 | ENSP00000264276 |
| DDR2 | PF07714 | 563 | 847 | Lapatinib | 1.576 | 0.01 | 0.024 | 0.05 | ENSP00000356899 |
| DDR2 | PF00069 | 564 | 845 | Lapatinib | 1.576 | 0.01 | 0.024 | 0.05 | ENSP00000356899 |
| PEAK1 | PF07714 | 1449 | 1656 | PHA-665752 | 0.146 | 0.007 | 0.024 | 0.043 | ENSP00000452796 |
| PEAK1 | PF00069 | 1456 | 1659 | PHA-665752 | 0.146 | 0.007 | 0.024 | 0.043 | ENSP00000452796 |
| AFF4 | IDR | 712 | 924 | PD-0325901 | 0.112 | 0.003 | 0.026 | 0.163 | ENSP00000265343 |
| ROCK2 | PF00069 | 92 | 354 | Nilotinib | 0.335 | 0.009 | 0.027 | 0.062 | ENSP00000317985 |
| MYO18B | PF00063 | 573 | 1207 | Irinotecan | 0.539 | 0.007 | 0.027 | 0.141 | ENSP00000386096 |
| RABEP1 | PF09311 | 612 | 807 | Nutlin-3 | 0.127 | 0.009 | 0.028 | 0.279 | ENSP00000262477 |
| TEC | PF00779 | 118 | 147 | PF2341066 | 2.793 | 0.007 | 0.028 | 0.161 | ENSP00000370912 |
| MYO3B | PF00063 | 355 | 1055 | PLX4720 | 2.186 | 0.002 | 0.028 | 0.022 | ENSP00000335100 |
| SPTAN1 | PF08726 | 2407 | 2475 | L-685458 | 2.07 | 0.008 | 0.029 | 0.088 | ENSP00000350882 |
| LAMA1 | PF02210 | 2743 | 2868 | PD-0332991 | 1.866 | 0.009 | 0.029 | 0.138 | ENSP00000374309 |
| LAMA1 | PF00054 | 2743 | 2872 | PD-0332991 | 1.866 | 0.009 | 0.029 | 0.138 | ENSP00000374309 |
| TEK | PF00069 | 825 | 1090 | AZD0530 | 0.337 | 0.008 | 0.03 | 0.165 | ENSP00000369375 |
| TEK | PF07714 | 824 | 1090 | AZD0530 | 0.337 | 0.008 | 0.03 | 0.165 | ENSP00000369375 |
| NCOA2 | IDR | 1125 | 1280 | Lapatinib | 2.638 | 0.004 | 0.03 | 0.149 | ENSP00000399968 |
| EXT1 | PF09258 | 480 | 729 | Nilotinib | 1.96 | 0.006 | 0.03 | 0.075 | ENSP00000367446 |
| MTOR | PF02259 | 1513 | 1908 | Nilotinib | 0.339 | 0.002 | 0.03 | 0.048 | ENSP00000354558 |
| IKZF3 | IDR | 149 | 248 | Paclitaxel | 0.733 | 0.007 | 0.03 | 0.168 | ENSP00000344544 |
| MTOR | PF02259 | 1513 | 1908 | PD-0332991 | 0.328 | 0.007 | 0.03 | 0.042 | ENSP00000354558 |
| NRAS | PF08477 | 5 | 119 | LBW242 | 1.451 | 0.005 | 0.031 | 0.028 | ENSP00000358548 |
| TSSK1B | PF07714 | 17 | 268 | Erlotinib | 2.531 | 0.003 | 0.032 | 0.21 | ENSP00000375081 |
| TSSK1B | PF00069 | 17 | 272 | Erlotinib | 2.531 | 0.003 | 0.032 | 0.21 | ENSP00000375081 |
| TNK2 | PF00069 | 190 | 442 | PD-0332991 | 0.092 | 0.004 | 0.034 | 0.052 | ENSP00000371341 |
| EPHA5 | PF01404 | 60 | 233 | Irinotecan | 0.554 | 0.008 | 0.036 | 0.015 | ENSP00000273854 |
| SUZ12 | PF09733 | 545 | 681 | L-685458 | 0.04 | 0.008 | 0.036 | 0.384 | ENSP00000316578 |
| GAB1 | IDR | 498 | 557 | PF2341066 | 2.394 | 0.008 | 0.036 | 0.643 | ENSP00000262995 |
| EHBP1 | IDR | 231 | 423 | ZD-6474 | 0.364 | 0.004 | 0.037 | 0.332 | ENSP00000263991 |
| CACNB2 | IDR | 500 | 660 | RAF265 | 0.502 | 0.009 | 0.038 | 0.234 | ENSP00000320025 |
| NF1 | PF00616 | 1256 | 1451 | TAE684 | 0.487 | 0.006 | 0.039 | 0.086 | ENSP00000351015 |
| GUCY2C | PF01094 | 54 | 384 | Irinotecan | 0.603 | 0.008 | 0.04 | 0.093 | ENSP00000261170 |
| HDAC4 | IDR | 76 | 288 | Nilotinib | 2.234 | 0.009 | 0.042 | 0.636 | ENSP00000264606 |
| PAPPA | Q13219.667.923 | 667 | 923 | AZD0530 | 0.257 | 0.006 | 0.044 | 0.059 | ENSP00000330658 |
| MYC | PF01056 | 16 | 360 | Irinotecan | 1.337 | 0.003 | 0.044 | 0.022 | ENSP00000367207 |
| MYH10 | PF00063 | 87 | 802 | AEW541 | 0.599 | 0.006 | 0.046 | 0.056 | ENSP00000353590 |
| NRK | PF00780 | 1214 | 1549 | Topotecan | 0.655 | 0.007 | 0.046 | 0.023 | ENSP00000438378 |
| Sep-06 | IDR | 293 | 457 | Erlotinib | 4.23 | 0.006 | 0.048 | 0.046 | ENSP00000378115 |
| NF1 | PF00616 | 1256 | 1451 | LBW242 | 0.297 | 0.007 | 0.048 | 0.04 | ENSP00000351015 |
| THRAP3 | IDR | 642 | 955 | Paclitaxel | 0.731 | 0.01 | 0.048 | 0.098 | ENSP00000346634 |
| RASA1 | IDR | 400 | 502 | PHA-665752 | 3.258 | 0.006 | 0.048 | 0.227 | ENSP00000274376 |
| FANCA | O15360.93.531 | 93 | 531 | ZD-6474 | 0.106 | 0.004 | 0.048 | 0.011 | ENSP00000373952 |
| ACACB | PF01039 | 1780 | 2333 | PLX4720 | 0.254 | 0.009 | 0.049 | 0.084 | ENSP00000367079 |
| NEK5 | IDR | 295 | 515 | Paclitaxel | 0.71 | 0.009 | 0.05 | 0.056 | ENSP00000347767 |
| MSH6 | PF00488 | 1075 | 1325 | RAF265 | 1.65 | 0.005 | 0.05 | 0.083 | ENSP00000234420 |
| GSG2 | IDR | 266 | 379 | 17-AAG | 0.606 | 0.008 | NA | 0.024 | ENSP00000325290 |
| MAK | PF07714 | 6 | 278 | 17-AAG | 0.696 | 0.005 | NA | 0.015 | ENSP00000313021 |
| MAK | PF00069 | 4 | 284 | 17-AAG | 0.696 | 0.005 | NA | 0.015 | ENSP00000313021 |
| ADARB2 | PF02137 | 408 | 731 | AEW541 | 0.385 | 0.006 | NA | 0.073 | ENSP00000370713 |
| RPS6KA2 | PF07714 | 441 | 692 | AEW541 | 1.594 | 0.007 | NA | 0.027 | ENSP00000422435 |
| RPS6KA2 | PF00069 | 440 | 697 | AEW541 | 1.594 | 0.007 | NA | 0.027 | ENSP00000422435 |
| ADARB2 | PF02137 | 408 | 731 | AZD6244 | 0.145 | 0.007 | NA | 0.028 | ENSP00000370713 |
| FANCA | O15360.93.531 | 93 | 531 | AZD6244 | 0.14 | 0.006 | NA | 0.026 | ENSP00000373952 |
| IL1R1 | PF01582 | 387 | 537 | AZD6244 | 2.815 | 0.007 | NA | 0.012 | ENSP00000386380 |
| LIMK1 | PF00069 | 308 | 564 | AZD6244 | 1.718 | 0.01 | NA | 0.011 | ENSP00000444452 |
| LIMK1 | PF07714 | 307 | 568 | AZD6244 | 1.718 | 0.01 | NA | 0.011 | ENSP00000444452 |
| LIMK1 | PF07714 | 371 | 632 | AZD6244 | 1.718 | 0.01 | NA | 0.011 | ENSP00000409717 |
| LIMK1 | PF00069 | 372 | 628 | AZD6244 | 1.718 | 0.01 | NA | 0.011 | ENSP00000409717 |
| MSH5 | PF00488 | 561 | 794 | AZD6244 | 0.201 | 0.006 | NA | 0.046 | ENSP00000431693 |
| MSH5- | PF00488 | 561 | 794 | AZD6244 | 0.201 | 0.006 | NA | 0.046 | ENSP00000417871 |
| SAPCD1 | | | | | | | | | |
| SIRT1 | IDR | 648 | 747 | AZD6244 | 0.029 | 0.004 | NA | 0.028 | ENSP00000212015 |
| ADARB2 | PF02137 | 408 | 731 | Erlotinib | 0.157 | 0.01 | NA | 0.148 | ENSP00000370713 |
| DYRK1B | PF07714 | 113 | 318 | Erlotinib | 3.035 | 0.007 | NA | 0.054 | ENSP00000469863 |
| LMTK2 | PF07714 | 138 | 406 | Erlotinib | 0.144 | 0.008 | NA | 0.02 | ENSP00000297293 |
| LMTK2 | PF00069 | 140 | 403 | Erlotinib | 0.144 | 0.008 | NA | 0.02 | ENSP00000297293 |
| MINK1 | IDR | 266 | 598 | Erlotinib | 2.275 | 0.002 | NA | 0.074 | ENSP00000347427 |
| NCKIPSD | Q9NZQ3.308.546 | 308 | 546 | Erlotinib | 2.151 | 0.009 | NA | 0.012 | ENSP00000294129 |
| RPS6KL1 | PF07714 | 200 | 523 | Erlotinib | 0.049 | 0.006 | NA | 0.017 | ENSP00000351086 |
| MAPK10 | PF07714 | 67 | 274 | Lapatinib | 3.493 | 0.008 | NA | 0.018 | ENSP00000352157 |
| MINK1 | IDR | 266 | 598 | Lapatinib | 1.876 | 0.007 | NA | 0.012 | ENSP00000347427 |
| MYO3A | PF00069 | 21 | 287 | Lapatinib | 2.273 | 0.004 | NA | 0.012 | ENSP00000265944 |
| MYO3A | PF07714 | 23 | 283 | Lapatinib | 2.315 | 0.008 | NA | 0.012 | ENSP00000265944 |
| PGBD3 | IDR | 252 | 590 | Lapatinib | 2.578 | 0.01 | NA | 0.031 | ENSP00000423550 |
| PSEN2 | IDR | 40 | 107 | Lapatinib | 2.805 | 0.008 | NA | 0.031 | ENSP00000375745 |
| ZMYND10 | 075800.213.377 | 213 | 377 | Lapatinib | 0.125 | 0.009 | NA | 0.081 | ENSP00000231749 |
| DYRK1A | PF07714 | 161 | 372 | Nutlin-3 | 0.108 | 0.009 | NA | 0.071 | ENSP00000381932 |
| DYRK1A | PF00069 | 159 | 479 | Nutlin-3 | 0.108 | 0.009 | NA | 0.071 | ENSP00000381932 |
| ITGA5 | PF08441 | 490 | 921 | Nutlin-3 | 1.655 | 0.009 | NA | 0.05 | ENSP00000293379 |
| MLK4 | PF07714 | 124 | 398 | Nutlin-3 | 0.203 | 0.008 | NA | 0.229 | ENSP00000355583 |
| MLK4 | PF00069 | 125 | 397 | Nutlin-3 | 0.203 | 0.008 | NA | 0.229 | ENSP00000355583 |
| MYH10 | IDR | 1421 | 1848 | Nutlin-3 | 0.265 | 0.004 | NA | 0.21 | ENSP00000353590 |
| PSKH2 | PF07714 | 66 | 278 | Nutlin-3 | 0.348 | 0.006 | NA | 0.033 | ENSP00000276616 |
| DTX1 | PF02825 | 23 | 94 | Paclitaxel | 0.552 | 0.009 | NA | 0.085 | ENSP00000257600 |
| CTBP2 | PF02826 | 680 | 863 | Panobinostat | 1.156 | 0.009 | NA | 0.014 | ENSP00000311825 |
| LAMP1 | PF01299 | 111 | 417 | Panobinostat | 1.23 | 0.001 | NA | 0.011 | ENSP00000333298 |
| RB1 | PF01858 | 373 | 573 | Panobinostat | 1.236 | 0.009 | NA | 0.093 | ENSP00000267163 |
| LIMK1 | PF00069 | 308 | 564 | PD-0325901 | 1.8 | 0.005 | NA | 0.014 | ENSP00000444452 |
| LIMK1 | PF07714 | 307 | 568 | PD-0325901 | 1.8 | 0.005 | NA | 0.014 | ENSP00000444452 |
| LIMK1 | PF07714 | 371 | 632 | PD-0325901 | 1.8 | 0.005 | NA | 0.014 | ENSP00000409717 |
| LIMK1 | PF00069 | 372 | 628 | PD-0325901 | 1.8 | 0.005 | NA | 0.014 | ENSP00000409717 |
| MSH5 | PF00488 | 561 | 794 | PD-0325901 | 0.257 | 0.009 | NA | 0.012 | ENSP00000431693 |
| MSH5- | PF00488 | 561 | 794 | PD-0325901 | 0.257 | 0.009 | NA | 0.012 | ENSP00000417871 |
| SAPCD1 | | | | | | | | | |
| REM1 | PF02421 | 82 | 249 | PD-0325901 | 0.353 | 0.008 | NA | 0.086 | ENSP00000201979 |
| MAPK10 | PF07714 | 67 | 274 | PD-0332991 | 2.545 | 0.008 | NA | 0.024 | ENSP00000352157 |
| ABL2 | PF00069 | 290 | 536 | PF2341066 | 0.361 | 0.003 | NA | 0.011 | ENSP00000427562 |
| ABL2 | PF07714 | 288 | 538 | PF2341066 | 0.361 | 0.003 | NA | 0.011 | ENSP00000427562 |
| CAMK2A | PF07714 | 15 | 264 | PF2341066 | 2.679 | 0.01 | NA | 0.014 | ENSP00000381412 |
| CAMK2A | PF00069 | 13 | 271 | PF2341066 | 2.679 | 0.01 | NA | 0.014 | ENSP00000381412 |
| ERBB3 | PF01030 | 56 | 166 | PF2341066 | 0.279 | 0.008 | NA | 0.056 | ENSP00000267101 |
| MYH11 | IDR | 1324 | 1979 | PF2341066 | 0.582 | 0.009 | NA | 0.039 | ENSP00000379616 |
| PRKG1 | PF00027 | 137 | 218 | PF2341066 | 0.205 | 0.005 | NA | 0.075 | ENSP00000363092 |
| TEC | IDR | 96 | 299 | PF2341066 | 2.381 | 0.008 | NA | 0.161 | ENSP00000370912 |
| MSH3 | PF05192 | 533 | 842 | PHA-665752 | 0.222 | 0.009 | NA | 0.152 | ENSP00000265081 |
| MYCL1 | PF01056 | 187 | 251 | PHA-665752 | 0.043 | 0.005 | NA | 0.031 | ENSP00000380494 |
| LIMK1 | PF00069 | 308 | 564 | PLX4720 | 2.481 | 0.003 | NA | 0.014 | ENSP00000444452 |
| LIMK1 | PF07714 | 307 | 568 | PLX4720 | 2.481 | 0.003 | NA | 0.014 | ENSP00000444452 |
| LIMK1 | PF07714 | 371 | 632 | PLX4720 | 2.481 | 0.003 | NA | 0.014 | ENSP00000409717 |
| LIMK1 | PF00069 | 372 | 628 | PLX4720 | 2.481 | 0.003 | NA | 0.014 | ENSP00000409717 |
| PIK3C2B | PF00613 | 812 | 988 | PLX4720 | 0.225 | 0.007 | NA | 0.044 | ENSP00000356155 |
| EML4 | PF03451 | 234 | 309 | RAF265 | 1.961 | 0.007 | NA | 0.017 | ENSP00000384939 |
| FGFR3 | PF00069 | 475 | 749 | RAF265 | 0.435 | 0.009 | NA | 0.06 | ENSP00000339824 |
| FGFR3 | PF07714 | 474 | 750 | RAF265 | 0.435 | 0.009 | NA | 0.06 | ENSP00000339824 |
| CARS | PF01406 | 128 | 535 | Sorafenib | 1.874 | 0.009 | NA | 0.058 | ENSP00000369897 |
| TRIM67 | PF00622 | 648 | 768 | TAE684 | 0.443 | 0.009 | NA | 0.022 | ENSP00000355613 |
| GUCY2F | PF01094 | 75 | 408 | TKI258 | 1.96 | 0.008 | NA | 0.102 | ENSP00000218006 |
| PIP5K1B | PF01504 | 148 | 433 | TKI258 | 1.509 | 0.008 | NA | 0.033 | ENSP00000435778 |
| PRKG2 | IDR | 665 | 762 | Topotecan | 0.257 | 0.005 | NA | 0.022 | ENSP00000264399 |
| RIOK2 | IDR | 268 | 468 | Topotecan | 0.284 | 0.008 | NA | 0.022 | ENSP00000283109 |
| ETV5 | PF04621 | 43 | 408 | ZD-6474 | 0.294 | 0.007 | NA | 0.038 | ENSP00000441737 |
| SIRT1 | IDR | 648 | 747 | ZD-6474 | 0.165 | 0.007 | NA | 0.171 | ENSP00000212015 |
| SUZ12 | Q15022.428.544 | 428 | 544 | ZD-6474 | 1.931 | 0.006 | NA | 0.322 | ENSP00000316578 |
| URB2 | Q14146.21.253 | 21 | 253 | ZD-6474 | 0.326 | 0.009 | NA | 0.186 | ENSP00000258243 |
| WNK1 | IDR | 2497 | 2588 | ZD-6474 | 0.263 | 0.004 | NA | 0.3 | ENSP00000433548 |
| ERLIN2 | PF01145 | 25 | 207 | 17-AAG | 0.616 | 0.009 | NC | 0.009 | ENSP00000276461 |
| GOLGA5 | PF09787 | 235 | 711 | 17-AAG | 1.253 | 0.008 | NC | 0.006 | ENSP00000163416 |
| MAPK10 | PF00069 | 64 | 359 | 17-AAG | 1.436 | 0.005 | NC | 0.005 | ENSP00000352157 |
| NRK | PF00780 | 1214 | 1549 | 17-AAG | 0.744 | 0.006 | NC | 0.007 | ENSP00000438378 |
| PRKG2 | PF07714 | 453 | 694 | 17-AAG | 0.676 | 0.003 | NC | 0 | ENSP00000264399 |
| PRKG2 | PF00069 | 454 | 711 | 17-AAG | 0.66 | 0.004 | NC | 0 | ENSP00000264399 |
| AFF4 | PF05110 | 2 | 1160 | AEW541 | 0.566 | 0.002 | NC | 0.002 | ENSP00000265343 |
| CIC | IDR | 968 | 1205 | AEW541 | 0.411 | 0.003 | NC | 0.007 | ENSP00000459719 |
| HSP90B1 | PF00183 | 257 | 783 | AEW541 | 0.506 | 0.007 | NC | 0.003 | ENSP00000299767 |
| NTSR1 | PF10323 | 97 | 381 | AEW541 | 1.882 | 0.003 | NC | 0.005 | ENSP00000359532 |
| NTSR1 | PF00001 | 80 | 364 | AEW541 | 1.659 | 0.005 | NC | 0.005 | ENSP00000359532 |
| ANGPTL4 | PF00147 | 185 | 399 | AZD0530 | 0.067 | 0.006 | NC | 0.008 | ENSP00000472551 |
| PDK1 | PF10436 | 56 | 240 | AZD0530 | 2.494 | 0.009 | NC | 0.002 | ENSP00000376352 |
| RHOA | PF08477 | 7 | 120 | AZD0530 | 1.605 | 0.005 | NC | 0.009 | ENSP00000400175 |
| RHOA | PF00071 | 7 | 179 | AZD0530 | 1.655 | 0.009 | NC | 0.009 | ENSP00000400175 |
| RHOA | PF00025 | 7 | 172 | AZD0530 | 1.655 | 0.009 | NC | 0.009 | ENSP00000400175 |
| RPS6KL1 | PF07714 | 200 | 523 | AZD0530 | 0.055 | 0.005 | NC | 0.001 | ENSP00000351086 |
| BRAF | PF07714 | 457 | 712 | AZD6244 | 2.174 | 0 | NC | 0 | ENSP00000288602 |
| BRAF | PF00069 | 458 | 712 | AZD6244 | 2.174 | 0 | NC | 0 | ENSP00000288602 |
| IFNG | PF00714 | 15 | 152 | AZD6244 | 0.092 | 0.009 | NC | 0.009 | ENSP00000229135 |
| KRAS | PF08477 | 5 | 119 | AZD6244 | 1.251 | 0 | NC | 0.001 | ENSP00000256078 |
| KRAS | PF00025 | 3 | 161 | AZD6244 | 1.223 | 0.001 | NC | 0.001 | ENSP00000256078 |
| KRAS | PF00071 | 5 | 164 | AZD6244 | 1.223 | 0.001 | NC | 0.001 | ENSP00000256078 |
| NRAS | PF08477 | 5 | 119 | AZD6244 | 1.859 | 0 | NC | 0 | ENSP00000358548 |
| NRAS | PF00071 | 5 | 164 | AZD6244 | 1.772 | 0 | NC | 0 | ENSP00000358548 |
| NRAS | PF00025 | 3 | 162 | AZD6244 | 1.772 | 0 | NC | 0 | ENSP00000358548 |
| NRAS | PF00009 | 45 | 163 | AZD6244 | 1.691 | 0 | NC | 0 | ENSP00000358548 |
| TIMP3 | PF00965 | 22 | 194 | AZD6244 | 2.046 | 0.006 | NC | 0.006 | ENSP00000266085 |
| FES | PF00069 | 563 | 812 | Erlotinib | 0.079 | 0.01 | NC | 0 | ENSP00000331504 |
| FES | PF07714 | 562 | 814 | Erlotinib | 0.079 | 0.01 | NC | 0 | ENSP00000331504 |
| MYO3B | IDR | 307 | 363 | Erlotinib | 1.599 | 0.005 | NC | 0.001 | ENSP00000335100 |
| RHOA | PF08477 | 7 | 120 | Erlotinib | 2.07 | 0.004 | NC | 0.006 | ENSP00000400175 |
| RHOA | PF00071 | 7 | 179 | Erlotinib | 1.972 | 0.006 | NC | 0.006 | ENSP00000400175 |
| RHOA | PF00025 | 7 | 172 | Erlotinib | 1.972 | 0.006 | NC | 0.006 | ENSP00000400175 |
| RHPN2 | PF03097 | 111 | 512 | Erlotinib | 0.31 | 0.006 | NC | 0.006 | ENSP00000254260 |
| STAR | PF01852 | 78 | 280 | Erlotinib | 0.127 | 0.006 | NC | 0.006 | ENSP00000276449 |
| CDC73 | Q6P1J9.1.108 | 1 | 108 | Irinotecan | 0.397 | 0.002 | NC | 0.003 | ENSP00000356405 |
| KRAS | PF08477 | 5 | 119 | Irinotecan | 0.829 | 0.001 | NC | 0.003 | ENSP00000256078 |
| KRAS | PF00025 | 3 | 161 | Irinotecan | 0.851 | 0.003 | NC | 0.003 | ENSP00000256078 |
| KRAS | PF00071 | 5 | 164 | Irinotecan | 0.851 | 0.003 | NC | 0.003 | ENSP00000256078 |
| LAMA1 | PF00054 | 2514 | 2657 | L-685458 | 3.149 | 0.009 | NC | 0.002 | ENSP00000374309 |
| LAMA1 | PF02210 | 2514 | 2653 | L-685458 | 3.149 | 0.009 | NC | 0.002 | ENSP00000374309 |
| P2RX7 | Q99572.510.595 | 510 | 595 | L-685458 | 2.744 | 0.001 | NC | 0.006 | ENSP00000442349 |
| P2RX7 | IDR | 558 | 595 | L-685458 | 2.942 | 0.001 | NC | 0.006 | ENSP00000442349 |
| BRAF | PF07714 | 457 | 712 | Lapatinib | 0.646 | 0.001 | NC | 0.001 | ENSP00000288602 |
| BRAF | PF00069 | 458 | 712 | Lapatinib | 0.646 | 0.001 | NC | 0.001 | ENSP00000288602 |
| COL1A1 | PF01410 | 1245 | 1463 | Lapatinib | 2.218 | 0.009 | NC | 0.003 | ENSP00000225964 |
| DFNA5 | PF04598 | 1 | 469 | Lapatinib | 2.356 | 0.004 | NC | 0.004 | ENSP00000386670 |
| MMP1 | PF00413 | 108 | 261 | Lapatinib | 0.209 | 0.004 | NC | 0.001 | ENSP00000322788 |
| RHOA | PF08477 | 7 | 120 | Lapatinib | 2.39 | 0.002 | NC | 0.005 | ENSP00000400175 |
| RHOA | PF00071 | 7 | 179 | Lapatinib | 2.222 | 0.005 | NC | 0.005 | ENSP00000400175 |
| RHOA | PF00025 | 7 | 172 | Lapatinib | 2.222 | 0.005 | NC | 0.005 | ENSP00000400175 |
| SPRY2 | PF05210 | 183 | 294 | Lapatinib | 3.461 | 0.002 | NC | 0.006 | ENSP00000366306 |
| ALPK1 | Q96QP1.43.507 | 43 | 507 | LBW242 | 2.197 | 0.008 | NC | 0.004 | ENSP00000177648 |
| ITGB8 | PF00362 | 54 | 469 | LBW242 | 1.587 | 0.008 | NC | 0.003 | ENSP00000222573 |
| PRCC | IDR | 255 | 491 | LBW242 | 3.654 | 0.008 | NC | 0.008 | ENSP00000271526 |
| ABL2 | PF00069 | 290 | 536 | Nilotinib | 0.295 | 0.006 | NC | 0.009 | ENSP00000427562 |
| ABL2 | PF07714 | 288 | 538 | Nilotinib | 0.295 | 0.006 | NC | 0.009 | ENSP00000427562 |
| CARS | PF01406 | 128 | 535 | Nilotinib | 1.726 | 0.01 | NC | 0.01 | ENSP00000369897 |
| CDK2 | PF00069 | 245 | 334 | Nilotinib | 0.214 | 0.01 | NC | 0.01 | ENSP00000452514 |
| CTDSPL | PF03031 | 107 | 266 | Nilotinib | 2.96 | 0.004 | NC | 0.004 | ENSP00000273179 |
| CLTC | PF00637 | 979 | 1119 | Nutlin-3 | 0.253 | 0.008 | NC | 0.006 | ENSP00000269122 |
| COL3A1 | PF01410 | 1249 | 1465 | Nutlin-3 | 0.222 | 0.004 | NC | 0.008 | ENSP00000304408 |
| CTDSPL | PF03031 | 107 | 266 | Nutlin-3 | 1.659 | 0.01 | NC | 0.01 | ENSP00000273179 |
| MAPKAPK5 | PF00069 | 25 | 304 | Nutlin-3 | 0.339 | 0.008 | NC | 0.008 | ENSP00000449381 |
| MAPKAPK5 | PF07714 | 23 | 296 | Nutlin-3 | 0.339 | 0.008 | NC | 0.008 | ENSP00000449381 |
| NOVA1 | PF00013 | 424 | 488 | Nutlin-3 | 0.113 | 0.008 | NC | 0.002 | ENSP00000438875 |
| RPS6KA2 | PF07714 | 441 | 692 | Nutlin-3 | 2.512 | 0.001 | NC | 0.002 | ENSP00000422435 |
| RPS6KA2 | PF00069 | 440 | 697 | Nutlin-3 | 2.512 | 0.001 | NC | 0.002 | ENSP00000422435 |
| STAT5B | PF02864 | 332 | 583 | Nutlin-3 | 0.196 | 0.001 | NC | 0 | ENSP00000293328 |
| TP53 | PF00870 | 95 | 288 | Nutlin-3 | 0.756 | 0 | NC | 0.001 | ENSP00000269305 |
| CDC73 | PF05179 | 233 | 525 | Paclitaxel | 0.591 | 0.008 | NC | 0.001 | ENSP00000356405 |
| CHRNA5 | PF02932 | 257 | 380 | Paclitaxel | 0.656 | 0.003 | NC | 0.002 | ENSP00000299565 |
| KRAS | PF08477 | 5 | 119 | Paclitaxel | 0.917 | 0.001 | NC | 0.003 | ENSP00000256078 |
| KRAS | PF00025 | 3 | 161 | Paclitaxel | 0.922 | 0.003 | NC | 0.003 | ENSP00000256078 |
| KRAS | PF00071 | 5 | 164 | Paclitaxel | 0.922 | 0.003 | NC | 0.003 | ENSP00000256078 |
| RET | PF07714 | 725 | 1005 | Paclitaxel | 0.679 | 0.008 | NC | 0.002 | ENSP00000347942 |
| RET | PF00069 | 726 | 1003 | Paclitaxel | 0.679 | 0.008 | NC | 0.002 | ENSP00000347942 |
| SLC14A1 | PF03253 | 113 | 417 | Paclitaxel | 0.705 | 0.009 | NC | 0.009 | ENSP00000412309 |
| TAB1 | PF00481 | 70 | 333 | Paclitaxel | 0.71 | 0.005 | NC | 0.005 | ENSP00000216160 |
| KRAS | PF08477 | 5 | 119 | Panobinostat | 0.916 | 0 | NC | 0 | ENSP00000256078 |
| KRAS | PF00025 | 3 | 161 | Panobinostat | 0.927 | 0 | NC | 0 | ENSP00000256078 |
| KRAS | PF00071 | 5 | 164 | Panobinostat | 0.927 | 0 | NC | 0 | ENSP00000256078 |
| NRAS | PF08477 | 5 | 119 | Panobinostat | 1.142 | 0 | NC | 0 | ENSP00000358548 |
| NRAS | PF00071 | 5 | 164 | Panobinostat | 1.132 | 0 | NC | 0 | ENSP00000358548 |
| NRAS | PF00025 | 3 | 162 | Panobinostat | 1.132 | 0 | NC | 0 | ENSP00000358548 |
| ADARB2 | PF02137 | 408 | 731 | PD-0325901 | 0.187 | 0.003 | NC | 0.007 | ENSP00000370713 |
| BRAF | PF07714 | 457 | 712 | PD-0325901 | 2.041 | 0 | NC | 0 | ENSP00000288602 |
| BRAF | PF00069 | 458 | 712 | PD-0325901 | 2.041 | 0 | NC | 0 | ENSP00000288602 |
| KRAS | PF08477 | 5 | 119 | PD-0325901 | 1.323 | 0 | NC | 0 | ENSP00000256078 |
| KRAS | PF00025 | 3 | 161 | PD-0325901 | 1.31 | 0 | NC | 0 | ENSP00000256078 |
| KRAS | PF00071 | 5 | 164 | PD-0325901 | 1.31 | 0 | NC | 0 | ENSP00000256078 |
| NRAS | PF08477 | 5 | 119 | PD-0325901 | 1.667 | 0 | NC | 0 | ENSP00000358548 |
| NRAS | PF00071 | 5 | 164 | PD-0325901 | 1.602 | 0 | NC | 0 | ENSP00000358548 |
| NRAS | PF00025 | 3 | 162 | PD-0325901 | 1.602 | 0 | NC | 0 | ENSP00000358548 |
| NRAS | PF00009 | 45 | 163 | PD-0325901 | 1.555 | 0 | NC | 0 | ENSP00000358548 |
| TP53 | PF00870 | 95 | 288 | PD-0325901 | 0.758 | 0.002 | NC | 0.005 | ENSP00000269305 |
| TRIM67 | PF00622 | 648 | 768 | PD-0325901 | 0.318 | 0.005 | NC | 0.002 | ENSP00000355613 |
| TTN | PF00041 | 27866 | 27946 | PD-0325901 | 0.176 | 0.006 | NC | 0.008 | ENSP00000467141 |
| GRK4 | PF00069 | 189 | 447 | PF2341066 | 1.818 | 0.007 | NC | 0.007 | ENSP00000381129 |
| GRK4 | PF07714 | 190 | 432 | PF2341066 | 1.818 | 0.007 | NC | 0.007 | ENSP00000381129 |
| MKNK1 | PF00069 | 52 | 374 | PF2341066 | 0.294 | 0.008 | NC | 0.008 | ENSP00000361014 |
| MYH9 | PF00063 | 83 | 764 | PF2341066 | 0.711 | 0.01 | NC | 0.003 | ENSP00000216181 |
| NRAS | PF00071 | 5 | 164 | PF2341066 | 1.269 | 0.006 | NC | 0.006 | ENSP00000358548 |
| NRAS | PF00025 | 3 | 162 | PF2341066 | 1.269 | 0.006 | NC | 0.006 | ENSP00000358548 |
| NRAS | PF08477 | 5 | 119 | PF2341066 | 1.281 | 0.008 | NC | 0.006 | ENSP00000358548 |
| RHOH | PF00025 | 4 | 164 | PF2341066 | 0.465 | 0.008 | NC | 0.004 | ENSP00000371219 |
| TP53 | PF00870 | 95 | 288 | PF2341066 | 0.853 | 0.004 | NC | 0.005 | ENSP00000269305 |
| CAMK4 | PF00069 | 46 | 300 | PHA-665752 | 2.733 | 0.006 | NC | 0.006 | ENSP00000282356 |
| CAMK4 | PF07714 | 47 | 288 | PHA-665752 | 2.733 | 0.006 | NC | 0.006 | ENSP00000282356 |
| CHRNA5 | PF02932 | 257 | 380 | PHA-665752 | 0.147 | 0.008 | NC | 0.002 | ENSP00000299565 |
| FES | PF00069 | 563 | 812 | PHA-665752 | 0.111 | 0.004 | NC | 0 | ENSP00000331504 |
| FES | PF07714 | 562 | 814 | PHA-665752 | 0.111 | 0.004 | NC | 0 | ENSP00000331504 |
| GRK4 | PF00069 | 189 | 447 | PHA-665752 | 2.786 | 0.002 | NC | 0.002 | ENSP00000381129 |
| GRK4 | PF07714 | 190 | 432 | PHA-665752 | 2.786 | 0.002 | NC | 0.002 | ENSP00000381129 |
| PRCC | IDR | 255 | 491 | PHA-665752 | 3.625 | 0.005 | NC | 0.005 | ENSP00000271526 |
| BRAF | PF07714 | 457 | 712 | PLX4720 | 4.016 | 0 | NC | 0 | ENSP00000288602 |
| BRAF | PF00069 | 458 | 712 | PLX4720 | 4.016 | 0 | NC | 0 | ENSP00000288602 |
| IRAK1 | PF00069 | 216 | 516 | PLX4720 | 5.098 | 0.006 | NC | 0.006 | ENSP00000358997 |
| IRAK1 | PF07714 | 216 | 515 | PLX4720 | 5.098 | 0.006 | NC | 0.006 | ENSP00000358997 |
| KRAS | PF08477 | 5 | 119 | PLX4720 | 0.538 | 0.006 | NC | 0.01 | ENSP00000256078 |
| KRAS | PF00025 | 3 | 161 | PLX4720 | 0.551 | 0.01 | NC | 0.01 | ENSP00000256078 |
| KRAS | PF00071 | 5 | 164 | PLX4720 | 0.551 | 0.01 | NC | 0.01 | ENSP00000256078 |
| BRAF | PF07714 | 457 | 712 | RAF265 | 1.391 | 0 | NC | 0 | ENSP00000288602 |
| BRAF | PF00069 | 458 | 712 | RAF265 | 1.391 | 0 | NC | 0 | ENSP00000288602 |
| EML4 | PF03451 | 234 | 309 | Sorafenib | 2.686 | 0.008 | NC | 0.005 | ENSP00000384939 |
| MAPK14 | PF00069 | 24 | 308 | Sorafenib | 1.714 | 0.007 | NC | 0.002 | ENSP00000229794 |
| NRAS | PF08477 | 5 | 119 | Sorafenib | 1.367 | 0.005 | NC | 0.006 | ENSP00000358548 |
| NRAS | PF00071 | 5 | 164 | Sorafenib | 1.34 | 0.006 | NC | 0.006 | ENSP00000358548 |
| NRAS | PF00025 | 3 | 162 | Sorafenib | 1.34 | 0.006 | NC | 0.006 | ENSP00000358548 |
| ETV1 | PF04621 | 29 | 347 | TAE684 | 0.507 | 0.004 | NC | 0.004 | ENSP00000384085 |
| OBSCN | PF07686 | 2906 | 2974 | TAE684 | 0.163 | 0.008 | NC | 0.005 | ENSP00000455507 |
| OBSCN | PF07679 | 2901 | 2982 | TAE684 | 0.163 | 0.008 | NC | 0.005 | ENSP00000455507 |
| ADCK1 | PF03109 | 136 | 252 | TKI258 | 0.589 | 0.007 | NC | 0.001 | ENSP00000238561 |
| ADCK1 | PF00069 | 154 | 337 | TKI258 | 0.615 | 0.008 | NC | 0.001 | ENSP00000238561 |
| GRK4 | PF00069 | 189 | 447 | TKI258 | 1.574 | 0.006 | NC | 0.006 | ENSP00000381129 |
| GRK4 | PF07714 | 190 | 432 | TKI258 | 1.574 | 0.006 | NC | 0.006 | ENSP00000381129 |
| KRAS | PF08477 | 5 | 119 | TKI258 | 0.733 | 0 | NC | 0 | ENSP00000256078 |
| KRAS | PF00025 | 3 | 161 | TKI258 | 0.749 | 0 | NC | 0 | ENSP00000256078 |
| KRAS | PF00071 | 5 | 164 | TKI258 | 0.749 | 0 | NC | 0 | ENSP00000256078 |
| ADCK1 | PF00069 | 154 | 337 | Topotecan | 0.69 | 0.007 | NC | 0.005 | ENSP00000238561 |
| CAMK2A | PF07714 | 15 | 264 | Topotecan | 2.079 | 0.003 | NC | 0.009 | ENSP00000381412 |
| CAMK2A | PF00069 | 13 | 271 | Topotecan | 2.079 | 0.003 | NC | 0.009 | ENSP00000381412 |
| CDC73 | Q6P1J9.1.108 | 1 | 108 | Topotecan | 0.36 | 0.01 | NC | 0.004 | ENSP00000356405 |
| KRAS | PF08477 | 5 | 119 | Topotecan | 0.835 | 0.001 | NC | 0.002 | ENSP00000256078 |
| KRAS | PF00025 | 3 | 161 | Topotecan | 0.852 | 0.002 | NC | 0.002 | ENSP00000256078 |
| KRAS | PF00071 | 5 | 164 | Topotecan | 0.852 | 0.002 | NC | 0.002 | ENSP00000256078 |
| MYC | PF01056 | 16 | 360 | Topotecan | 1.346 | 0 | NC | 0.002 | ENSP00000367207 |
| NRAS | PF00071 | 5 | 164 | Topotecan | 1.192 | 0.009 | NC | 0.009 | ENSP00000358548 |
| NRAS | PF00025 | 3 | 162 | Topotecan | 1.192 | 0.009 | NC | 0.009 | ENSP00000358548 |
| PRCC | IDR | 255 | 491 | Topotecan | 1.752 | 0.009 | NC | 0.009 | ENSP00000271526 |
| ACVR1B | PF00069 | 209 | 533 | ZD-6474 | 0.347 | 0.005 | NC | 0.001 | ENSP00000442656 |
| PTPN1 | PF00102 | 40 | 276 | ZD-6474 | 0.346 | 0.008 | NC | 0.008 | ENSP00000360683 |
| SUFU | PF12470 | 252 | 473 | ZD-6474 | 0.296 | 0.007 | NC | 0.002 | ENSP00000358918 |
| ULK1 | PF07714 | 19 | 272 | ZD-6474 | 1.774 | 0.008 | NC | 0.001 | ENSP00000324560 |
| ULK1 | PF00069 | 18 | 278 | ZD-6474 | 1.774 | 0.008 | NC | 0.001 | ENSP00000324560 |

Those skilled in the art will recognize, or be able to ascertain using no more than routine experimentation, many equivalents to the specific embodiments of the method and compositions described herein. Such equivalents are intended to be encompassed by the following claims.

1. A method of treating a disease, the method comprising
treating a subject having the disease and identified as having genetic features in a drug-specific set of protein units with a compound identified as a protein unit-specific compound for the drug-specific set of protein units, wherein the disease is a protein unit-associated disease for the drug-specific set of protein units,
wherein the drug-specific set of protein units is a set of protein units where genetic features in the set of protein units are correlated with an effect of the compound, wherein the effect is a disease-associated effect for the disease, wherein the compound is a disease-associated compound for the disease, wherein the disease is a protein unit-associated disease for the drug-specific set of protein units,
wherein at least one of the protein units in the drug-specific set of protein units is a PFR or a PFR group of a protein, wherein genetic features in the PFR or PFR group of the protein are correlated with an effect of the compound but where genetic features in the protein as a whole are not correlated with the effect of the compound.

2. The method of claim 1, wherein the set of protein units consists of a single PFR for a protein.

3. The method of claim 1, wherein the disease is cancer, wherein the disease-associated effect is an anticancer effect, wherein the genetic features in the drug-specific set of protein units are present in one or more cancer cells of the subject.

4. The method of claim 1, wherein prior to treatment the subject is identified as having one or more cells having the genetic features in the drug-specific set of protein units.

5. The method of claim 1 further comprising, prior to treatment, detecting the genetic features in the drug-specific set of protein units in one or more cells of the subject.

6. The method of claim 3, wherein the cells are disease-related cells for the disease.

7. A method of identifying a drug-specific set of protein units for a compound and a disease, the method comprising
assessing correlation between genetic features in a test set of protein units and the effect of a compound on a disease, wherein at least one of the protein units in the test set of protein units is a PFR or a PFR group of a protein, wherein identification of a correlation between genetic features in the test set of protein units and the effect of the compound on a disease identify the test set of protein units as a drug-specific set of protein units for the compound and for the disease and identify the compound as a protein unit/disease-associated compound for the disease and for the test set of protein units.

8. A method of identifying protein unit-specific compounds for a set of protein units and a disease, the method comprising
assessing correlation between genetic features in a set of protein units and the effect of a test compound on a disease, wherein identification of a correlation between genetic features in the set of protein units and the effect of the test compound on a disease identify the test compound as a protein unit-specific compound for the set of protein units and for the disease and identify the set of protein units as a drug-specific set of protein units for the disease and for the test compound.

9. The method of claim 7, wherein the test set of protein units comprises at least one PFR and at least one whole protein.

10. The method of claim 7, wherein the test set of protein units comprises at least two PFRs.

11. The method of claim 7, wherein the test set of protein units comprises at least one PFR group.

12. The method of claim 7, wherein the test set of protein units consists of a single PFR for a protein, wherein the method further comprises assessing correlation between genetic features of the protein as a whole and the effect of the compound on the disease, wherein identification of a correlation between genetic features in the PFR for the protein and the effect of the compound on a disease and a lack of correlation between genetic features of the protein as a whole and the effect of the compound on the disease identify the PFR of the protein as a drug-specific PFR for the compound and for the disease and identify the compound as a PFR/disease-associated compound for the disease and for the PFR of the protein.

13. The method of claim 8, wherein the set of protein units consists of a single PFR for a protein, wherein the method further comprises assessing correlation between genetic features of the protein as a whole and the effect of the test compound on the disease, wherein identification of a correlation between genetic features in the PFR of the protein and the effect of the test compound on a disease and a lack of correlation between genetic features of the protein as a whole and the effect of the test compound on the disease identify the test compound as a PFR-specific compound for the PFR of the protein and for the disease and identify the PFR of the protein as a drug-specific PFR for the disease and for the test compound.

14. The method of claim 7, wherein identification of the correlations is accomplished by
identifying protein units in proteins,
categorizing genetic features by protein unit, wherein the genetic features are present or not present in disease-related cells,
categorizing the genetic features by whether the compound has the effect on the disease in subjects having the disease and having the genetic features or by whether the compound has the effect on the disease-related cells affected by the disease and having the genetic features, and
calculating the level of correlation between genetic features in the protein units and the effect of the compound.

15. The method of claim 14 further comprising calculating the level of correlation between genetic features in proteins as a whole and the effect of the compound.

16. The method of claim 14, wherein the disease-related cells are cancer cell lines, wherein the genetic features are categorized by whether the compound has the effect on the cancer cell lines having the genetic features.

17. A method of contributing to improving the effectiveness of a treatment of a disease in a population of subjects that have the disease, the method comprising
treating a subject having genetic features in a drug-specific set of protein units in one or more disease-related cells with a protein unit-specific compound for the set of protein units and for the disease and refraining from treating a subject that does not have genetic features in one or more members of the drug-specific set of protein units of one or more disease-related cells with the protein unit-specific compound,
wherein the drug-specific set of protein units is a set of protein units where genetic features in the set of protein units are correlated with an effect of the compound, wherein the effect is a disease-associated effect for the disease, wherein the compound is a disease-associated compound for the disease, wherein the disease is a protein unit-associated disease for the drug-specific set of protein units,
wherein at least one of the protein units in the set of drug-specific protein units is a PFR or a PFR group of a protein, wherein genetic features in the PFR or PFR group of the protein are correlated with an effect of the compound but where genetic features in the protein as a whole are not correlated with the effect of the compound.

18. The method of claim 17, wherein the set of protein units consists of a single PFR for a protein.

19. The method of claim 17, wherein the disease is cancer, wherein the disease-associated effect is an anticancer effect, wherein the genetic features in the drug-specific set of protein units is present in one or more cancer cells of the subject.

20. The method of claim 17, wherein prior to treatment the subject is identified as having one or more cells having the genetic features in the drug-specific set of protein units.

21. The method of claim 17 further comprising, prior to treatment, detecting the genetic features in the drug-specific set of protein units in one or more cells of the subject.

22. The method of claim 19, wherein the cells are disease-related cells for the disease.

23. A method of treating cancer, the method comprising
treating a subject having cancer and identified as having a genetic feature in a drug-specific PFR with a PFR-specific compound for the drug-specific PFR,
wherein the drug-specific PFR and PFR-specific compound for the drug-specific PFR are selected from one of the following pairs:

| Drug-Specific PFR | Compound |
|---|---|
| Amino acids 1245 to 1508 of MAP3K1 | Lapatinib |
| Amino acids 1246 to 1503 of MAP3K1 | Lapatinib |
| Amino acids 123 to 407 of MSH6 | AEW541 |
| Amino acids 280 to 460 of CACNB2 | L-685458 |
| Amino acids 148 to 248 of ADAM22 | TKI258 |
| Amino acids 1818 to 2102 of TPR | ZD-6474 |
| Amino acids 334 to 699 of AFF4 | PD-0325901 |
| Amino acids 76 to 288 of HDAC4 | Sorafenib |
| Amino acids 137 to 218 of PRKG1 | Sorafenib |
| Amino acids 38 to 151 of DAPK1 | PHA-665752 |
| Amino acids 1221 to 1309 of ITGB4 | TAE684 |
| Amino acids 2514 to 2657 of LAMA1 | AEW541 |
| Amino acids 2514 to 2653 of LAMA1 | AEW541 |
| Amino acids 28254 to 28339 of TTN | Topotecan |
| Amino acids 1442 to 1492 of MTOR | Topotecan |
| Amino acids 520 to 703 of PIK3CA | AEW541 |
| Amino acids 252 to 322 of DAPK1 | PLX4720 |
| Amino acids 814 to 1266 of SETDB1 | PF2341066 |
| Amino acids 814 to 1266 of SETDB1 | TAE684 |
| Amino acids 2514 to 2657 of LAMA1 | PF2341066 |
| Amino acids 2514 to 2653 of LAMA1 | PF2341066 |
| Amino acids 644 to 733 of DPYD | TKI258 |
| Amino acids 172 to 406 of MAP3K13 | RAF265 |
| Amino acids 171 to 406 of MAP3K13 | RAF265 |
| Amino acids 190 to 442 of TNK2 | TKI258 |
| Amino acids 4468 to 4599 of LRP1B | Sorafenib |
| Amino acids 748 to 903 of CDH2 | 17-AAG |
| Amino acids 1846 to 2050 of PI4KA | PD-0325901 |
| Amino acids 1818 to 2102 of TPR | TKI258 |
| Amino acids 980 to 1244 of INSRR | PD-0332991 |
| Amino acids 980 to 1244 of INSRR | PD-0332991 |
| Amino acids 28254 to 28339 of TTN | Lapatinib |
| Amino acids 60 to 233 of EPHA5 | Nutlin-3 |
| Amino acids 334 to 699 of AFF4 | AZD6244 |
| Amino acids 1 to 68 of MYC | AZD0530 |
| Amino acids 1345 to 1639 of CREBBP | AZD6244 |
| Amino acids 667 to 923 of PAPPA | LBW242 |
| Amino acids 28254 to 28339 of TTN | Nilotinib |
| Amino acids 979 to 1119 of CLTCL1 | TAE684 |
| Amino acids 32 to 108 of PIK3CA | AEW541 |
| Amino acids 816 to 1002 of GUCY2C | PHA-665752 |
| Amino acids 76 to 288 of HDAC4 | TKI258 |
| Amino acids 897 to 1184 of MECOM | ZD-6474 |
| Amino acids 1068 to 1217 of BCR | TAE684 |
| Amino acids 1 to 172 of SMG1 | LBW242 |
| Amino acids 1044 to 1233 of TIAM1 | L-685458 |
| Amino acids 30721 to 30807 of TTN | RAF265 |
| Amino acids 4993 to 5069 of TTN | PF2341066 |
| Amino acids 4990 to 5059 of TTN | PF2341066 |
| Amino acids 1083 to 1222 of BIRC6 | Nutlin-3 |
| Amino acids 148 to 248 of ADAM22 | Nilotinib |
| Amino acids 279 to 373 of PPARGC1A | Panobinostat |
| Amino acids 1695 to 1822 of TG | Panobinostat |
| Amino acids 1 to 68 of MYC | TAE684 |
| Amino acids 2694 to 2748 of CSMD3 | PD-0325901 |
| Amino acids 32714 to 32792 of TTN | AZD0530 |
| Amino acids 1125 to 1280 of NCOA2 | Erlotinib |
| Amino acids 807 to 1069 of PTK7 | PD-0325901 |
| Amino acids 695 to 878 of ALS2 | Panobinostat |
| Amino acids 114 to 294 of CTTN | ZD-6474 |
| Amino acids 622 to 697 of TNN | AEW541 |
| Amino acids 586 to 808 of BAI3 | AZD0530 |
| Amino acids 134 to 413 of EXT2 | TAE684 |
| Amino acids 2971 to 3050 of TTN | Topotecan |
| Amino acids 26686 to 26766 of TTN | 17-AAG |
| Amino acids 60 to 162 of ADAM12 | Irinotecan |
| Amino acids 492 to 561 of CPNE5 | AZD0530 |
| Amino acids 274 to 367 of TSSK1B | TAE684 |
| Amino acids 561 to 794 of MSH5 | ZD-6474 |
| Amino acids 561 to 794 of MSH5-SAPCD1 | ZD-6474 |
| Amino acids 303 to 334 of TNNI3K | AEW541 |
| Amino acids 521 to 605 of PCDH15 | Irinotecan |
| Amino acids 2054 to 2236 of MLL3 | Lapatinib |
| Amino acids 3718 to 3754 of LRP2 | PLX4720 |
| Amino acids 737 to 1068 of UBE3B | Panobinostat |
| Amino acids 7795 to 7885 of TTN | Topotecan |
| Amino acids 280 to 460 of CACNB2 | AZD0530 |
| Amino acids 137 to 218 of PRKG1 | TAE684 |
| Amino acids 1916 to 2020 of NAV3 | 17-AAG |
| Amino acids 87 to 802 of MYH10 | TAE684 |
| Amino acids 220 to 389 of NLRP3 | PD-0332991 |
| Amino acids 1711 to 2049 of CNTRL | TAE684 |
| Amino acids 1409 to 1488 of TAF1L | Panobinostat |
| Amino acids 824 to 916 of PCDH15 | Nutlin-3 |
| Amino acids 817 to 925 of CUBN | Nilotinib |
| Amino acids 1224 to 1458 of PTPRT | Paclitaxel |
| Amino acids 1649 to 1795 of FANCM | Nutlin-3 |
| Amino acids 769 to 942 of RASA1 | PF2341066 |
| Amino acids 87 to 802 of MYH10 | AZD0530 |
| Amino acids 947 to 1234 of GRIN2A | AZD6244 |
| Amino acids 50 to 94 of PLCG1 | PHA-665752 |
| Amino acids 40 to 140 of PLCG1 | PHA-665752 |
| Amino acids 410 to 617 of ZNF608 | Lapatinib |
| Amino acids 807 to 1069 of PTK7 | AZD6244 |
| Amino acids 199 to 527 of HIPK2 | TKI258 |
| Amino acids 190 to 442 of TNK2 | Nutlin-3 |
| Amino acids 31 to 186 of ADAMTS20 | AZD0530 |
| Amino acids 914 to 1030 of AATK | Lapatinib |
| Amino acids 382 to 604 of PAXIP1 | RAF265 |
| Amino acids 538 to 699 of MSH6 | Lapatinib |
| Amino acids 555 to 638 of SMO | 17-AAG |
| Amino acids 75 to 408 of GUCY2F | LBW242 |
| Amino acids 249 to 426 of RASGRF2 | Paclitaxel |
| Amino acids 524 to 607 of ROBO2 | PHA-665752 |
| Amino acids 400 to 545 of ACOXL | AZD0530 |
| Amino acids 645 to 739 of GTSE1 | PF2341066 |
| Amino acids 1 to 68 of MYC | AZD6244 |
| Amino acids 190 to 442 of TNK2 | ZD-6474 |
| Amino acids 46 to 188 of ALK | Panobinostat |
| Amino acids 512 to 728 of GUCY1A2 | LBW242 |
| Amino acids 1256 to 1451 of NF1 | Panobinostat |
| Amino acids 1249 to 1465 of COL3A1 | PHA-665752 |
| Amino acids 1 to 87 of SRPK1 | Lapatinib |
| Amino acids 21 to 253 of URB2 | RAF265 |
| Amino acids 320 to 391 of PRKD3 | ZD-6474 |
| Amino acids 47 to 157 of INSRR | Lapatinib |
| Amino acids 712 to 924 of AFF4 | PD-0325901 |
| Amino acids 92 to 354 of ROCK2 | Nilotinib |
| Amino acids 573 to 1207 of MYO18B | Irinotecan |
| Amino acids 612 to 807 of RABEP1 | Nutlin-3 |
| Amino acids 118 to 147 of TEC | PF2341066 |
| Amino acids 2407 to 2475 of SPTAN1 | L-685458 |
| Amino acids 2743 to 2868 of LAMA1 | PD-0332991 |
| Amino acids 2743 to 2872 of LAMA1 | PD-0332991 |
| Amino acids 825 to 1090 of TEK | AZD0530 |
| Amino acids 824 to 1090 of TEK | AZD0530 |
| Amino acids 1125 to 1280 of NCOA2 | Lapatinib |
| Amino acids 480 to 729 of EXT1 | Nilotinib |
| Amino acids 149 to 248 of IKZF3 | Paclitaxel |
| Amino acids 17 to 268 of TSSK1B | Erlotinib |
| Amino acids 17 to 272 of TSSK1B | Erlotinib |
| Amino acids 190 to 442 of TNK2 | PD-0332991 |
| Amino acids 545 to 681 of SUZ12 | L-685458 |
| Amino acids 498 to 557 of GAB1 | PF2341066 |
| Amino acids 231 to 423 of EHBP1 | ZD-6474 |
| Amino acids 500 to 660 of CACNB2 | RAF265 |
| Amino acids 1256 to 1451 of NF1 | TAE684 |
| Amino acids 54 to 384 of GUCY2C | Irinotecan |
| Amino acids 76 to 288 of HDAC4 | Nilotinib |
| Amino acids 667 to 923 of PAPPA | AZD0530 |
| Amino acids 87 to 802 of MYH10 | AEW541 |
| Amino acids 642 to 955 of THRAP3 | Paclitaxel |
| Amino acids 400 to 502 of RASA1 | PHA-665752 |
| Amino acids 1780 to 2333 of ACACB | PLX4720 |
| Amino acids 295 to 515 of NEK5 | Paclitaxel |
| Amino acids 1075 to 1325 of MSH6 | RAF265 |
| Amino acids 408 to 731 of ADARB2 | AEW541 |
| Amino acids 408 to 731 of ADARB2 | Erlotinib |
| Amino acids 113 to 318 of DYRK1B | Erlotinib |
| Amino acids 266 to 598 of MINK1 | Erlotinib |
| Amino acids 213 to 377 of ZMYND10 | Lapatinib |
| Amino acids 161 to 372 of DYRK1A | Nutlin-3 |
| Amino acids 159 to 479 of DYRK1A | Nutlin-3 |
| Amino acids 124 to 398 of MLK4 | Nutlin-3 |
| Amino acids 125 to 397 of MLK4 | Nutlin-3 |
| Amino acids 1421 to 1848 of MYH10 | Nutlin-3 |
| Amino acids 23 to 94 of DTX1 | Paclitaxel |
| Amino acids 373 to 573 of RB1 | Panobinostat |
| Amino acids 82 to 249 of REM1 | PD-0325901 |
| Amino acids 56 to 166 of ERBB3 | PF2341066 |
| Amino acids 137 to 218 of PRKG1 | PF2341066 |
| Amino acids 96 to 299 of TEC | PF2341066 |
| Amino acids 533 to 842 of MSH3 | PHA-665752 |
| Amino acids 475 to 749 of FGFR3 | RAF265 |
| Amino acids 474 to 750 of FGFR3 | RAF265 |
| Amino acids 128 to 535 of CARS | Sorafenib |
| Amino acids 75 to 408 of GUCY2F | TKI258 |
| Amino acids 648 to 747 of SIRT1 | ZD-6474 |
| Amino acids 428 to 544 of SUZ12 | ZD-6474 |
| Amino acids 21 to 253 of URB2 | ZD-6474 |
| Amino acids 2497 to 2588 of WNK1 | ZD-6474 |

24. The method of claim 23, wherein the genetic feature in the drug-specific PFR is present in one or more cancer cells of the subject.

25. The method of claim 23, wherein prior to treatment the subject is identified as having one or more cells having the genetic feature in the drug-specific PFR.

26. The method of claim 23 further comprising, prior to treatment, detecting the genetic feature in the drug-specific PFR in one or more cells of the subject.

27. The method of claim 1, wherein the each genetic feature is either the presence of one or more genetic alterations or a lack of one or more genetic alterations.
